(19) United States
(12) Patent Application Publication
(10) Pub. No.: US 2002/0013573 A1
(43) Pub. Date: Jan. 31, 2002

(54) APPARATUS AND METHOD FOR TRACKING AND COMPENSATING FOR EYE MOVEMENTS

(76) Inventors: WILLIAM B. TELFAIR, SAN JOSE, CA (US); PAUL R. YODER JR., NORWALK, CT (US); CARSTEN BEKKER, SAN RAMON, CA (US)

Correspondence Address: GEORGE N. CHACLAS, ATTORNEY, CUMMINGS, & LOCKWOOD, GRANITE SQUARE, 700 STATE STREET P.O. BOX 1960, NEW HAVEN, CT, 06509-1960, US

(21) Appl. No.: 08/969,128

(22) Filed: Nov. 11, 1997

Related U.S. Application Data
(63) Continuation-in-part of application No. 08/549,385, filed on Oct. 27, 1995, now Pat. No. 5,782,822.

(51) Int. Cl. A61B 18/20

(52) U.S. Cl. 606/5; 606/10

(57) ABSTRACT

A system for facilitating tracking of a moving object is disclosed. The object has a feature associated therewith which is illuminated with ambient light. The system includes illumination means for illuminating at least the feature of the object with a tracking light. The system also includes detection means for detecting an image of the feature and for outputting signals corresponding to movement of the image. The signals have a first component due to the tracking light and a second component due to the ambient light. Further, the system includes filter means for filtering the second component from the signals and for outputting the first component of the signals so that the ambient light is discriminated from the tracking light and the moving object can be tracked using the first component of the signals. In one embodiment, in which the object is a human eye, the system may also include logic means for receiving the signals and for generating tracking signals based thereon and means for directing a laser beam upon the eye based on the tracking signals to maintain a substantially centered condition between the optical axis of the laser beam and the visual axis of the eye.

### REFERENCE TO RELATED APPLICATION

This application is a continuation-in-part of co-pending patent application Ser. No. 08/549,385, filed on Oct. 27, 1995.

### BACKGROUND OF THE INVENTION

The present invention generally relates to a system and method for tracking a moving object. More specifically, this invention relates to a system and method for tracking movement of an eye during diagnostic analysis or during a surgical procedure wherein a laser beam is directed on the eye, and compensating for such movement so as to maintain a substantially centered condition between the laser beam and the eye.

Surgical procedures are known which aim to correct refractive disorders of a human eye through ablation of the cornea of the eye using laser radiation. Such procedures include Photorefractive Keratectomy (PRK), Phototherapeutic Keratectomy (PTK), and Laser In Situ Keratomileusis (LASIK). Typically, according to these procedures, laser pulses are scanned in sequence over centralized circular areas of the cornea to cause localized tissue ablation (what may be called “scanning laser” ablation) or are used to simultaneously irradiate similar centralized circular areas of the cornea (commonly referred to as wide area ablation). The treated areas are typically between 6 and 9 mm in diameter.

Scanning laser systems for use in corneal surgery were taught, for example, by L'Esperance in U.S. Pat. No. 4,665,913 and by Lin. in U.S. Pat. No. 5,520,679. Both of these patents deal with methods using 193 nm wavelength radiation from an excimer laser. An alternative scanning system invokes a photospallation mechanism to perform corneal ablation using a mid-infrared laser as described in U.S. patent application Ser. No. 08/549,385, of which the present application is a continuation-in-part.

Typically, the above-referenced (and other) scanning techniques for corneal sculpting involve rapidly moving a relatively small spot of laser radiation over a specific central portion of the corneal surface in a predefined pattern. This allows selective removal of tissue at various points within the scanned region, thereby cumulatively re-shaping the surface of the cornea into the desired geometry in a predictable fashion.

A problem which has plagued the art is that, during corneal refractive surgery, the eye which is receiving the laser pulses is subject to various involuntary and voluntary movements. The movements of the eye vary in type and in degree and may occur simultaneously. For example, one type of involuntary eye movement is known as a “saccade”. Saccades generally involve rapid eyeball rotations of up to 600 deg/sec and occur typically on a 10-30 msec time scale with amplitudes ranging from 1 to 10 degrees. See Bahill et al, Invest. Ophthalm. Vis. Sci., 21, 116, 198 1. A second type of involuntary eye movement involves tremors. Tremors may occur at rates of 10 to 200 Hz and with amplitudes on the order of 0.5 arc min. See Carpenter, Movements of the Eyes, 2^{nd} ed., 1988 and Findlay, “Frequency Analysis of Human Involuntary Eye Movement”, Kybernetik, 8, 207, 1971. Another type of involuntary eye movement involves drifts which can occur at velocities of about 4 arc min/sec and with significantly larger amplitudes than tremors. See Ditchburn, Eye Movements and Visual Perception, 1973. Studies of eye movements, such as one reported by Bahil et al (referenced above), indicate that extremely high accelerations of up to 40,000 deg/sec² may be involved in the fastest movements.

Eye movements often lead to misalignments, i.e., decentrations, of all or portions of the ablated region on the cornea. The treatment area decentrations are particularly harmful in the above mentioned surgical procedures since they may result in irregular astigmatism, glare phenomena, decreased visual acuity and lower contrast sensitivity. Such eye movements cause uneven distribution of tissue ablation patterns and must be minimized in order to achieve requisite surface smoothness. Implementation of such improved means for suppressing eye motion, while important in wide area ablation, is especially important in scanning laser delivery systems, which require precise execution of specific scanning algorithms, and spot placement accuracy on the order of 5 to 50 μm.

It is standard practice during corneal laser surgery for the patient's head to be securely restrained so movements of the eye being treated result only from roll of the eyeball within its socket. These movements cause the center of the cornea to shift position in the vertical and/or horizontal directions, usually by no more than 5 mm.

In some prior art apparatus for corneal surgery, the eyeball itself is further immobilized by clamping, suction rings or other means, such as stitching the eye to an eyelid retractor (called a speculum), such as that disclosed in U.S. Pat. No. 5,556,417 to Scher, so as to suppress movements of the eye. However, ever this further immobilization of the eye is not completely effective in suppressing all involuntary eye movements. These physical constraints also may be uncomfortable for the patient and may lead to infection, as in the case where invasive techniques such as stitching are used. The availability of a technique for tracking movements of the eye and compensating therefor would eliminate the need for immobilization of the eye during laser surgery.

Means for tracking an object typically involve an optical system for imaging the object or a portion thereof onto some form of sensor such as a video camera or an array of light detectors. It is essential that the object be illuminated so the image is sufficiently bright for detection. It is important for this tracking illumination to come from a source or sources under the control of the operator so that factors such as intensity, color, propagation direction, etc. can be optimized. Other sources, such as room lights, are not so optimized hence any light from these extraneous sources which reaches the image sensor will tend to obscure the ability of the tracker to sense the motion of the object.

Certain prior art techniques for tracking eye movement are based on pattern recognition of various features in the eye, such as localized variations in iris coloration or the circular shape of the pupil. These techniques are fundamentally digital in nature. For example, U.S. Pat. No. 5,231,678 to Cleveland et al teaches a digital method for detecting the edges of the pupil and analytically locating the pupil's center in reference to the first Purkinje point (the reflection from the anterior surface of the cornea). Other techniques rely on different reference points or alternative features of the eye. Because these techniques are digital, they require point-by-point acquisition of target features using video cameras and frame grabbers, as well as complex edge detection algorithms and sophisticated signal processing methods.

In such techniques, the response of the tracking system is limited by the video scanning rate of 60 Hz. This rate is not sufficient for tracking the fastest eye movements and also translates into an electronically complex system due to high sampling rate requirements which leave less than a millisecond for processing the signals. Furthermore, techniques predicated upon digital correlation processing of video signals derived from an optical image are often deficient due to unfavorable trade-offs between image size (or field of view) and spatial resolution due to limits on pixel size. In view of the foregoing, it is readily apparent that such digital techniques are unattractive for addressing the needs of refractive corneal laser surgery.

Other techniques for providing eye tracking are based on optical point trackers, such as the system taught by Crane and Steele in U.S. Pat. No. 4,287,410 and by Crane et al in U.S. Pat. No. 4,443,075. These systems utilize the lens-like properties of the eye to compare the displacements, over time, of the first and fourth Purkinje points (the latter is the reflection from the rear surface of the lens). These techniques purport to be able to distinguish between rotational and translational movements of the eye and to possess, in principle, sufficient speed to follow the fastest eye movements. Importantly, however, they cannot be utilized in conjunction with a surgical laser device which aims to modify the very anterior surface of the cornea which provides the specular reflection forming the first Purkinje point. Since the fourth Purkinje point is observed through the corneal surface, it would be severely degraded by the surgical intervention and hence rendered useless as a tracking aid. Even for diagnostic applications, the high eye-illuminating light levels needed to distinguish the low-reflectance fourth Purkinje point may provide unacceptable interference with other illumination means used in such diagnosis.

Yet other prior art techniques rely on tracking of the outer or inner edge of the iris, by detecting light scattered from such naturally occurring boundaries of the eye to measure differences in illumination from such boundaries. Such “differential reflection techniques”, as they are sometimes known, have the advantage of allowing for analog signal processing techniques which are known to be simpler, faster and have higher accuracy than the above-mentioned digital techniques.

One such naturally occurring boundary for use with differential reflection techniques is the limbus, which is the approximately circular intersection of the eye's transparent cornea with the translucent and white-colored sclera. The limbus also corresponds to the outer boundary of the colored iris which can be seen through the cornea. The limbus is a particularly attractive tracking landmark for corneal surgery, constituting, as it does, an integral part of the eyeball structure itself. It moves in the same manner as the central cornea area which is to be modified surgically, yet is located far enough away from the surgical site as not to interfere with the surgical procedure itself or for that procedure to affect the tracking landmark.

Such differential reflection prior art arrangements have been successful in sensing horizontal eye movements over a wide range of 15-25 degrees. However, sensing movements along the eye's vertical axis has been especially troublesome due to partial obscuration of the limbus by the upper and lower eye lids. One approach to overcoming this difficulty was disclosed by Knopp et al in PCT Patent Application Serial No. WO94/18883. The Knopp application teaches a differential light reflection technique using off-axis illumination of the eye and a pair of position sensors, each consisting of a multiplicity of segments. The sensors detect and measure both horizontal and vertical displacements of the limbus by continuously monitoring variations in the relative image illumination among the various segments. The technique taught by Knopp suffers from a major problem—that is, it does not provide the same high sensitivity in the vertical direction as in the horizontal direction. This is due to the much smaller differentials between illuminated areas on the detector elements produced by small vertical displacements as compared with those differentials produced by equivalent displacements in the horizontal direction. The resulting lower sensitivity characteristics of the system in the vertical displacement direction make the technique taught by Knopp difficult to implement in practice and reduce its ability to respond to small eye movement in the vertical direction. Furthermore, the disclosure of Knopp et al does not appreciate complications due to spurious signals which may be generated by ambient illumination or specular reflections from the eye. Such spurious signals may be especially troublesome when the eye is subject to off-axis illumination, which off-axis illumination is taught by Knopp et al.

Alternative differential reflection techniques use the pupil, which is the aperture in the iris, as the feature to be tracked. For e example, the technique taught by Cornsweet et al in U.S. Pat. No. 5,410,376 uses a quadrant detector to sense saccadic movement of the eye in both the vertical and horizontal directions. However, the technique of pupil backlighting taught by Cornweet et al requires illumination from a direction nearly coincident with the axis of the eye. Thus, this illumination would necessarily pass through the central area on the cornea. Since this region on the cornea is precisely that which would be ablated during PRK, PTK, or LASIK, the technique taught by Cornsweet et al would not be compatible with use during those surgical procedures relating to the cornea. Similarly, the pupil tracking methods taught by Taboada and Robinson in U.S. Pat. No. 5,345,281 are deficient for use with corneal surgical procedures in that they also rely upon nearly on-axis illumination of the eye through the region to be ablated on the cornea.

Still another differential reflection technique is taught by Frey et al in U.S. Pat. No. 5,632,742. In that reference, the eye is tracked using a natural feature, such as the limbus or the pupil of the eye, or a circular ink mark manually added thereto. The tracking is accomplished using a single light source focused to a plurality of positions on the feature of choice. By temporally sequencing the light pulses, a single detector can be used for sensing differences between light reflected or scattered from the various locations on the eye, such differences being indicative of eye movement in two orthogonal directions. This technique of Frey is limited in its dynamic range by the sizes of the illuminated light spots on the eye since the desired proportional error signal at each sampled location can be derived only while the chosen feature of the eye (inner or outer edge of the iris or the ink mark) lies within the appropriate spot. As described, the technique taught by Frey et al would also require fast signal detection, i.e., in less than 1 msec response time. While present technology can track such fast detection, such means are typically more costly and add complexity to the system by imposing stricter signal processing requirements. Further, the technique of Frey et al is sensitive to ambient illumination which may reach the eye and be reflected into the detector where it would tend to reduce detectability of the light pulses.

In view of the above, what is needed is a system and method for tracking movement from eye in both the horizontal and vertical directions which is fully compatible with laser surgery procedures, has fast response, and is insensitive to ambient illumination.

### SUMMARY OF THE INVENTION

One aspect of the present invention is directed to a system for facilitating tracking of a moving object. The object has a feature, associated therewith which is illuminated with ambient light. The system includes illumination means for illuminating at least the feature of the object with a tracking light. The system also includes detection means for detecting an image of the feature and for outputting signals corresponding to movement of the image. The signals have a first component due to the tracking light and a second component due to the ambient light. Further, the system includes filter means for filtering the second component from the signals and for outputting the first component of the signals so that the ambient light is discriminated from the tracking light and the moving object can be tracked using the first component of the signals.

Another aspect of the present invention is directed toward a system for compensating for movement of an eye of a patient during a surgical procedure. The eye has a feature and a visual axis associated therewith, wherein the feature is illuminated with ambient light. The surgical procedure includes directing a laser beam upon the eye using a mirror. The laser beam has an optical axis associated therewith. The system includes illumination means for illuminating at least the feature of the object with a tracking light. The system also includes detection means for detecting an image of the feature and for outputting signals corresponding to movement of the image, wherein the signals have a first component due to the tracking light and a second component due to the ambient light. A filter means is also included for filtering the second component from the signals and for outputting the first component of the signals so that the ambient light is discriminated from the tracking light. The system further includes logic means for receiving the filtered signals and for generating tracking signals based thereon. The system also includes means for directing the laser beam upon the eye based on the tracking signals to maintain a substantially centered condition between the optical axis of the laser beam and the visual axis of the eye.

### BRIEF DESCRIPTION OF THE DRAWINGS

Representative embodiments of the present invention will be described with reference to the following figures:

FIGS. 1(a) and 1(b) are diagrammatic views of the present invention.

FIG. 2 illustrates an alternative embodiment of the tracking light source 1005.

FIG. 3 illustrates the overlapping pattern of the light beams 1480 on the eye 900.

FIG. 4 is a diagrammatic view of the detector 1580.

FIG. 5(a) illustrates an image of the eye in an aligned position with respect to the detector elements 1620A-1620D.

FIG. 5(b) illustrates an image of the eye in an unaligned position with respect to the detector elements 1620A-1620D.

FIG. 6 is a diagrammatic view of the filter 1780.

FIG. 7a illustrates a means for adjusting the size of the tracking feature image and the detector 1580.

FIGS. 7b and 7c show an embodiment of the means for adjusting the size of the tracking feature image.

FIG. 7d shows an image of the eye in which the image size has not been adjusted.

FIG. 7e shows an image of the eye in which the image size has been adjusted.

FIG. 7f depicts an embodiment of the tracker subsystem 4000, including the means for adjusting the size of the image.

FIG. 8 is a block diagram of the system 1000 into which the present invention has been integrated.

FIG. 9 is a diagram of the system 1000 in which the laser subsystem 2000 and the microscope subsystem 3000 are shown in detail.

FIGS. 10a and 10b are an expanded schematic diagram and a detail view of embodiments of components shown in FIG. 9.

FIG. 11 illustrates components of the eye tracking subsystem 4000 for use in the system 1000.

FIGS. 12a and 12b are an expanded schematic diagram and a detail view of embodiments of components shown in FIG. 11.

FIG. 13 is a block diagram showing the interrelationship of the laser subsystem 2000 and the eye tracking subsystem 4000 that allows compensation for eye movements.

### DETAILED DESCRIPTION OF THE PREFERRED EMBODIMENTS

Reference is now made to the accompanying Figures for the purpose of describing, in detail, the preferred embodiments of the present invention. The Figures and accompanying detailed description are provided as examples of the invention and are not intended to limit the scope of the claims appended hereto.

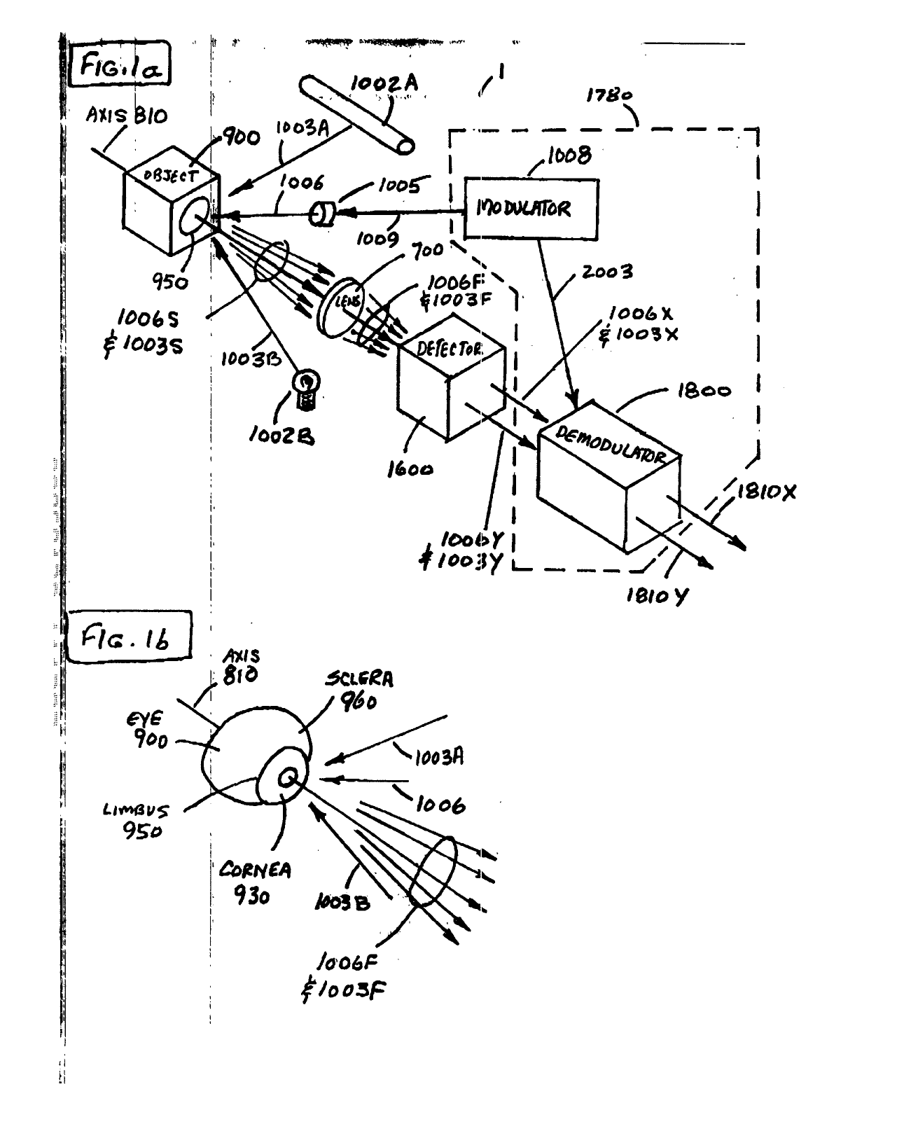

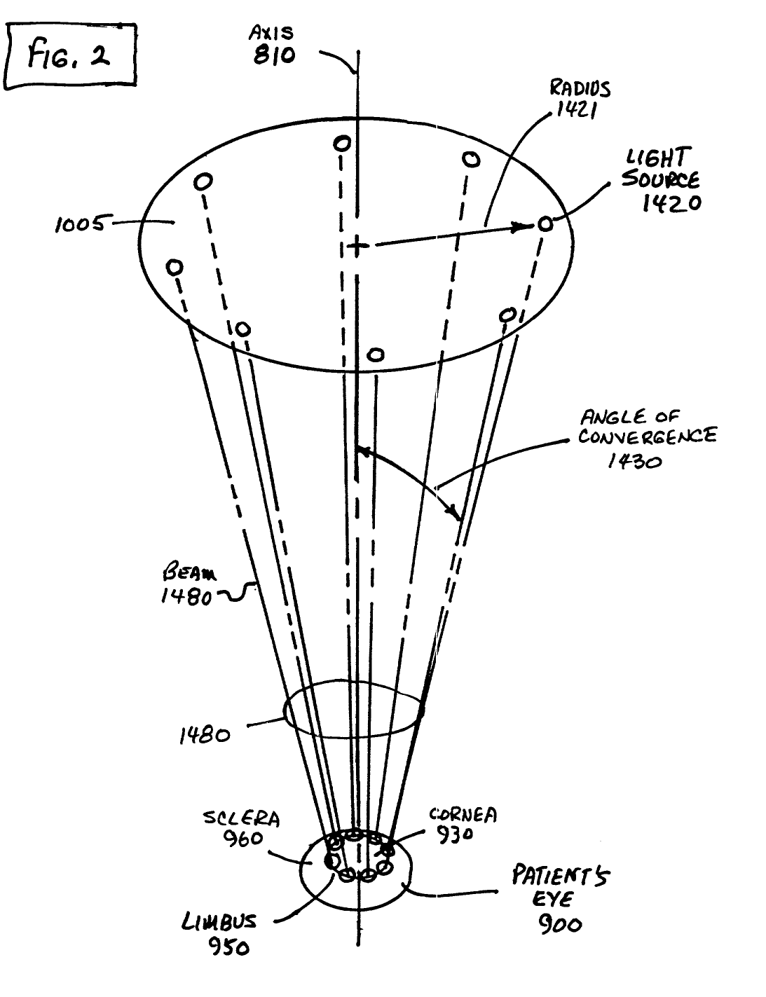

FIG. 1a depicts a system 1 for facilitating tracking of a moving object. Also shown there is a multiplicity of potential ambient light sources 1002A and 1002B that, without the system and method of the present invention, would interfere detrimentally with the tracking of the moving object. Ambient light sources such as fluorescent or incandescent room lights, microscope illuminators, or lights used for photography (including flash lamps) all can radiate light onto the object. Part of this ambient illumination is reflected or scattered by the object into and through the lens 700 of the tracker and can irradiate the detector 1600 of that tracker. This portion of the irradiation at the detector is called “stray light”. In general, this stray light will generate an electrical signal that may not be well correlated with the movement of the object. A portion of the illumination from the tracker illuminator 1005 also reflects or scatters from the object 900 into and through the lens 700 of the tracker and irradiates said detector array. This we term “signal light” because the electrical signal it generates is correlated with the object motion. Superposition of stray light onto the signal light reduces the signal-to-noise ratio at the detector array by increasing the noise level. Any reduction in signal-to-noise ratio will tend to interfere with the tracking system's ability to track the object.

In one embodiment of the present invention and as shown in FIG. 1b, the object to be tracked is a human eye 900. Tracking movement of the eye 900 is particularly useful during laser surgical procedures on the cornea 930 and diagnostic applications involving the eye. Eye tracking also is useful during automated refractometry or measurement of corneal topography. However, while the following description is set forth for tracking movement of the eye 900, it is contemplated that the present invention may be readily adapted for tracking the movement of other objects. For example, the system and method disclosed herein may be used to track the movement of objects such as selected skin tissue site relative to a dermatology surgical laser, an object of interest relative to a robotic machine vision system, or a docking site on a spacecraft relative to a remotely controlled probe. In each case, the object would bear a distinctive natural or artificial fiduciary marking to facilitate the tracking function.

Referring to FIG. 1b, as is well known, the eye 900 includes a transparent cornea 930 and a translucent and white-colored sclera 960. The eye 900 also includes a limbus 950, which is the intersection of the cornea 930 and the sclera 960. The limbus 950 also corresponds to the outer boundary of the colored iris of the eye 900 (not shown), which can be seen through the cornea 930 and is a characteristic feature of all human eyes.

The present invention uses a differential reflection technique to track the movement of the eye 900. Thus, the system and method disclosed herein involve detecting light scattered from the region of a naturally occurring feature of the eye 900 to measure differences in illumination from that region. The naturally occurring feature of the eye 900 should be substantially circular in shape; larger in diameter than the site to be surgically treated, i.e., 10 to 14 mm in diameter; constitute a boundary between sub-regions having differences in light reflectivity of at least 10 percent; and fixed to the object of interest, i.e., the eye. The naturally occurring feature of the eye 900 that is used for tracking the movement of the eye 900 may also be referred to herein as the “tracking feature.”

In our preferred embodiment, the limbus 950 is used as the tracking feature to track the movement of the eye 900. The limbus 950 is a desirable tracking feature because it is an integral part of the eyeball structure itself. Also, the limbus 950 moves in the same manner as the central area of the cornea 930. Thus, in frontal view, transition at the circular limbus 950 from the colored or tinted circular area and the white sclera 960 offers photometric contrast due to significant differences in light reflectivity of the iris and sclera in an axi-symmetric feature of the eye 900 that lends itself to tracking by the means described herein. Alternatively, the movement of the eye 900 may be tracked with reference to other naturally occurring boundaries of the eye 900, including the pupil, as well as non-natural features, such as colored ink markings on the sclera 960.

The light scattered from the eye 900 that will be detected is illustrated in FIG. 1a as light rays 1006S. The light rays 1006S are generated from light that is incident on a predetermined portion of the eye 900. The predetermined portion of the eye 900 includes the tracking feature and the areas surrounding the tracking feature. Thus, in the embodiment in which the limbus 950 is the tracking feature, the predetermined portion of the eye 900 that is illuminated is the iris (not shown) inside the cornea 930, the limbus 950, and the sclera 960 surrounding the limbus 950.

As is seen in FIG. 1a, the tracking light source 1005 and the ambient light sources 1002 provide light that is incident on the predetermined portion of the eye 900 to generate the superimposed light rays 1003S and 1006S. The tracking light source 1005 receives synchronization signals 1009 from modulator 1008. The tracking light source 1005 illuminates the predetermined portion of the object 900 with the tracking illumination 1006 based on the synchronization signals 1009. Thus, the synchronization signals 1009 cause the tracking light source 1005 to illuminate the predetermined portion of the eye 900 at a predetermined frequency, which differs from the frequency of the ambient light rays 1003A and 1003B originating at any of the stray light sources 1002A and 1002B.

The ambient light sources 1002A and 1002B represents one or more light sources that deliver unwanted light 1003A and 1003B to the predetermined portion of eye 900. The ambient light source 1002 thus is any light source, aside from the tracking light source 1005, that may be present when the system or method of the present invention is practiced. For example, the ambient light source 1002A or 1002B may be an illuminator associated with a microscope or ambient room illumination. Some ambient light sources 1002A or 1002B, such as fluorescent lights, typically have a known frequency, such as 60 or 120 Hz, which is significantly different from the predetermined frequency, of, for example, 200 to 300 Hz, at which the tracking light source 1005 illuminates the predetermined portion of the eye 900. Others, like tungsten bulbs, produce continuous illumination.

Thus, from the above, it is seen that the tracking illumination 1006 and the ambient light 1003A and 1003B are incident on, and scatter from, the predetermined portion of the eye 900 to generate the light rays 1003S and 1006S. As such, the light rays 1003S and 1006S may be viewed as two superimposed components. The first component is due to the tracking illumination 1006 and the second is due to the ambient light 1003.

The system 1 also includes a lens 700 that is positioned to receive the light rays 1003S and 1006S and to focus them in the form of light rays 1003F and 1006F onto certain detector elements of the detector 1600. In this way, an image 1670 (not shown) of the tracking feature, here, the limbus 950, is formed at and can be detected by the detector 1600. The image 1670 may be viewed as having two components, one due to the tracking illumination 1006 and the other due to the ambient light 1003A and 1003B.

The detector 1600 outputs detector signals 1006X and 1003X which are characterized by eye movement in the X direction and 1006Y and 1003Y which relate to eye movement in the Y direction. The detector signals output from the detector 1600 may be therefore viewed as having two X components and two Y components, the first due to the tracking illumination 1006 and the second due to the ambient light 1003A and 1003B.

As will be described in more detail below, the demodulator 1800 filters the signals and outputs only the component of the detector signals corresponding to the tracking illumination 1006 as tracking signals 1810X and 1810Y. The demodulator 1800 rejects the second component of the detector signals that are due to the ambient light 1003. By synchronizing the tracking light source 1005 to the synchronizing signals 1009, the tracker is rendered insensitive to the stray light originating at ambient light sources 1002A and 1002B and ultimately imaged upon detector 1600. Since the, signals 1810 output from filter 1780 result only from light received from tracking light source 1005, variations in intensity, color, direction of propagation, etc. in ambient light source 1002 do not compromise the operation of the tracker system. The signal-to-noise ratios of the output signals 1810X and 1810Y are thus substantially increased as is the tracker's ability to track the eye. The tracking signals 1810 may be used to track the movement of the eye 900, for example, by using them to measure the lateral displacement of the apex of the eyeball while performing visual tasks such as reading or observing a video screen. These measurements may be of interest to visual scientists studying the behavior of the human eye. Other ways, in which the present eye tracker might prove valuable would be in measuring the ability of the human eye to follow rapid motions of targets in simulated military encounters or in measuring eye displacements during exposure to accelerations during flight training.

The tracking light source 1005 and its illumination of the eye 900 is next described in more detail. The tracking light source 1005 generates tracking illumination 1006, which illuminates the predetermined portion of the eye 900 substantially uniformly.

In one embodiment, the tracking light source 1005 is an individual light generating element that is positioned to illuminate the portion of the eye 900 from a substantially axial direction with respect to the axis a 810. For example, the individual light generating element may be positioned a few degrees off of the axis 810 so that the predetermined portion of the eye 900 is substantially uniformly illuminated with light. In this embodiment, the individual light generating element may comprise a light emitting diode (LED), a diode laser, or the like. We prefer that the individual light generating element emit monochromatic light at a near-infrared wavelength of about 0.88 μm because of its low visibility to the human eye.

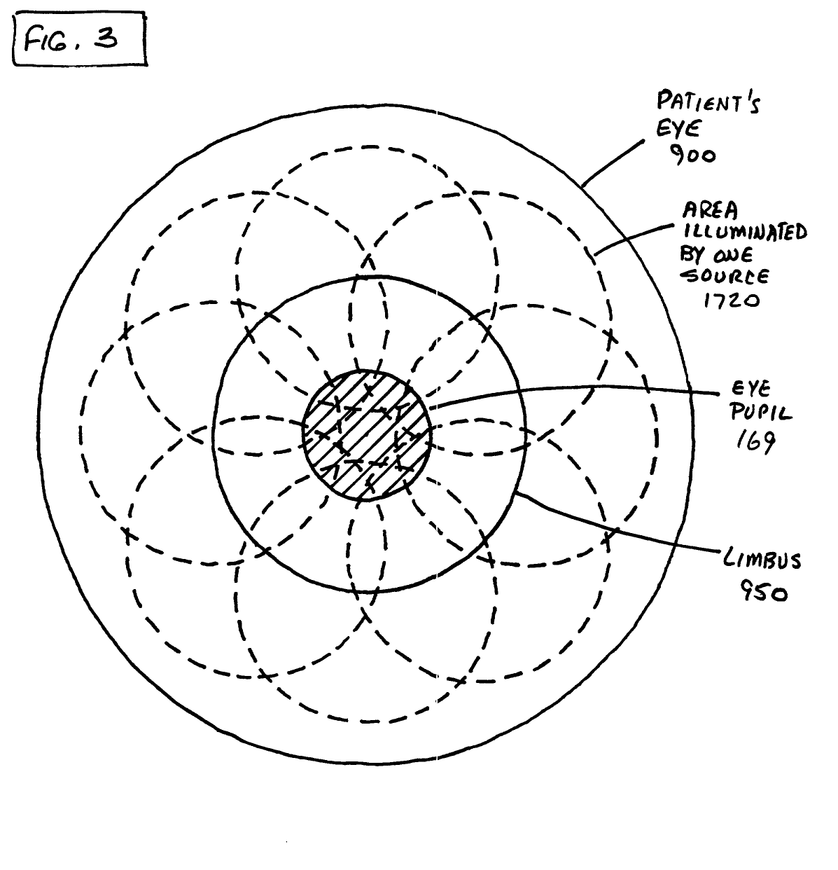

An alternative embodiment of the tracking light source 1005 is depicted in FIG. 2. There, it is seen that the tracking light source 1005 may comprise a plurality (e.g., 8) of individual light generating elements 1420. The individual light generating elements 1420 are positioned in a ring-like manner, equidistant from, and off of, the axis 810. Each of the plurality of elements 1420 generates a light beam 1480 which illuminates a predetermined portion of the eye 900 as follows.

In this alternative embodiment, the predetermined portion of the eye 900 is illuminated such that a light beam 1480 from one of the plurality of elements 1420 overlaps with light beams 1480 from adjacent elements 1420. Thus, as shown in FIG. 2 and more clearly in FIG. 3, each light beam 1480 from an element 1420 illuminates an area 1720 on the predetermined portion of the eye 900. This overlap and the radial extent of the illuminated region of the eye 900 ensure that the predetermined portion of the eye 900 remains substantially uniformly illuminated when it moves.

If the radius 1421 of the ring of sources 1420 is a significant fraction of their distances to the eye 900, the angle of convergence 1430 of the beams 1480 with respect to the axis 810 may be large enough that the beams 1480 striking the cornea 930 or the nearby sclera 960 might reflect specularly into the lens 700. This light might then reach the detector elements of detector 1600 and adversely affect the performance of the tracker.

To illustrate, in an embodiment of the invention with light sources 1420 at an angle 1430 of approximately 56 degrees to the axis 810 (FIG. 2), rays specularly reflected from the human cornea 930 of radius of curvature approximately 8 mm would be imaged within 2 mm square detector elements if the magnification produced by lens 700 is about unity and an array of detector elements is located 6.35 mm from the axis 810. This specularly-reflected light would be superimposed upon the image of the tracking feature and, being brighter than the scattered light from the eye 900, would adversely affect the ability of the tracker to measure true eye movements. If the sources 1420 were to be moved significantly closer to the axis 810, this potential problem is reduced or alleviated. For instance, using the example just described, the spurious images reflected specularly from the cornea or the adjacent stroma would not be seen by the detector elements if the sources were located no more than 30 degrees off the axis 810.

The importance of using near-coaxial illumination in avoiding interference from spurious signals due to specular reflections has not been appreciated by some of the prior art, including the methods represented by PCT Application No. WO 94/18883 due to Knopp et al. The dual light source arrangement therein described may not be symmetric enough to ensure illumination uniformity over the full predetermined area of the eye. Further, it may allow substantial interference from specular reflections that enter the detector means thereby degrading measurement of the eye's motions.

The tracker illumination subsystem utilized in a pupil-tracking version of the present invention would need to be designed so that the specularly-reflected light therefrom does not interfere with the tracking function. This design would follow the principles just described for the case of the limbus-tracking system. The light sources 1420 should, in a design for tracking a pupil, be located no more than 10 degrees off the axis 810.

In the alternative embodiment of tracking light source 1005 as depicted in FIG. 2, the wavelength of the light beams 1480 from the elements 1420 are chosen to lie in the near-infrared range of wavelength approximately 0.8 to 1.0 μm. To achieve this, light-emitting diodes (LEDs), such as the DPI-E805 type units manufactured by Photonic Detectors, Inc., may be used. We prefer to use such a wavelength because the sensitivity of the human eye is extremely low at the 0.88 μm emission wavelength of these devices so the observed intensity of any portion of the light beam 1480 reflected or scattered by a cornea surface will be so small as not to affect observation of the patient's eye by the surgeon. In addition, because of its low visibility to the human eye, the light beams 1480 will not interfere with fixation of the eye 900 by the patient upon a visible light target located within a fixation target device.

The tracking light source 1005 is modulated at a predefined frequency. This is done using the synchronization signals 1009 received from the modulator 1008. More specifically, the modulator 1008 varies the synchronization signals 1009 between zero and X volts at the predefined frequency. The value of X is the maximum operating voltage of the tracking light source 1005.

In yet another embodiment of the tracking light source 1005, the light beam 1006 many emanate from a tungsten filament lamp that provides for both visual observation of the eye 900 and tracking the movement of the eye 900. This beam 1006 would be intensity modulated by a mechanical chopping device such as the type available from Oriel Corporation as their Model 75155 Enclosed Optical Chopper with motor-driven, 30-aperture, slotted wheel. This chopping device is capable of modulating the beam 1006 at frequencies up to 3000 Hz. The light beam 1006 would appear to be of constant intensity to the surgeon's eye so it would serve well for visual alignment of the eye 900 to an alignment reference (reticle) pattern in the microscope.

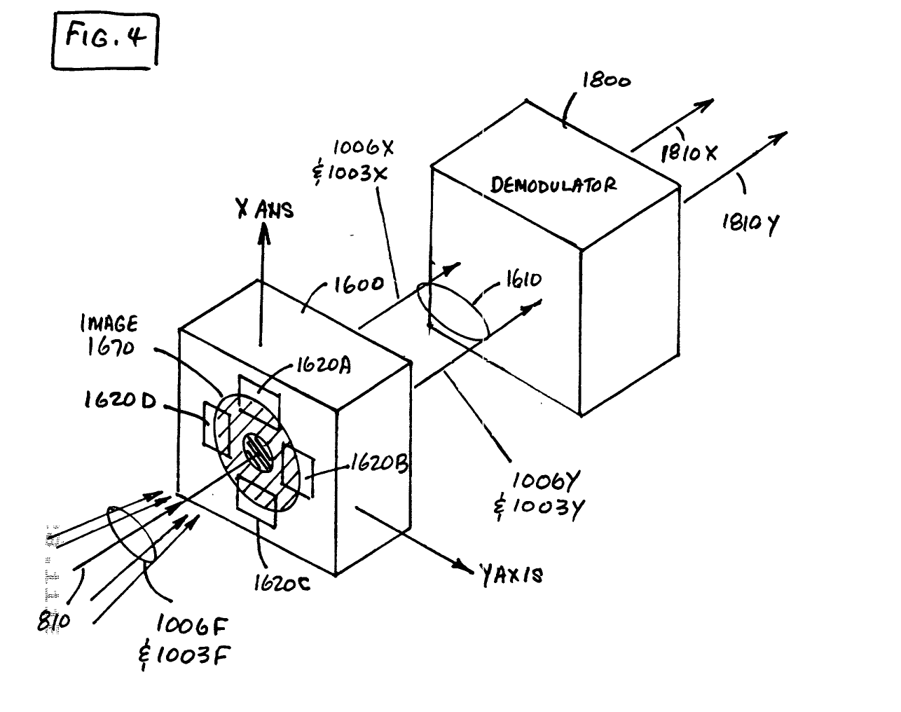

Referring next to FIG. 4, the detector 1600 is described in more detail. As is seen there, the detector 1600 receives the light rays 1006F and 1003F, which collectively form image 1670, and outputs the detector signals 1006X, 1003X, 1006Y and 1003Y which collectively are designated as 1610. The detector 1600 outputs signals 1610 to the demodulator 1800 and then to amplifier 1700 (not shown).

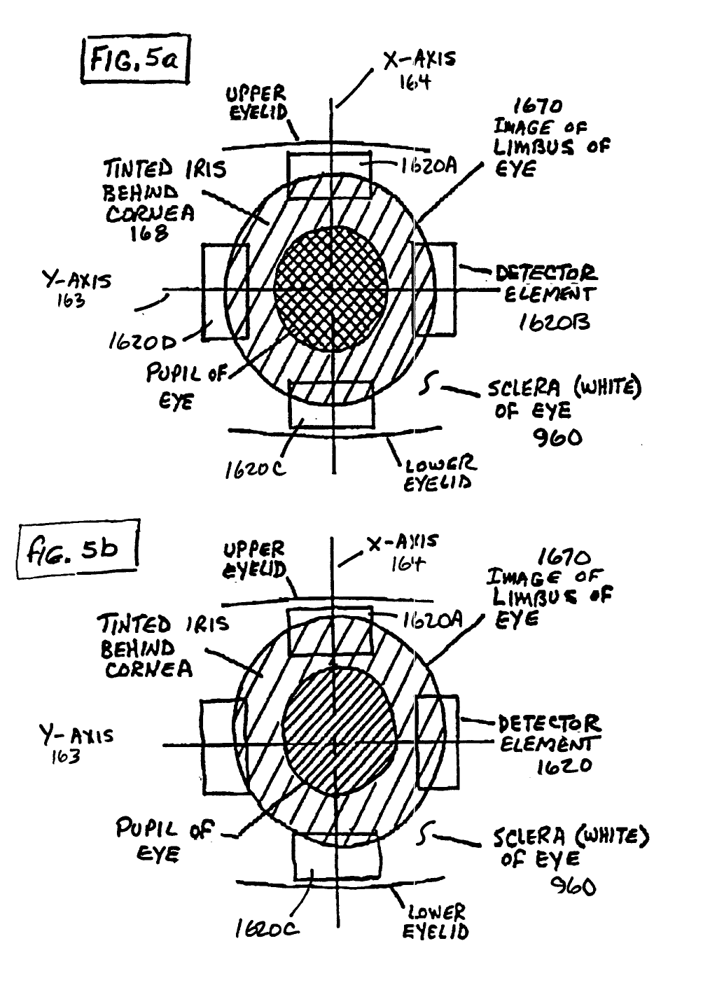

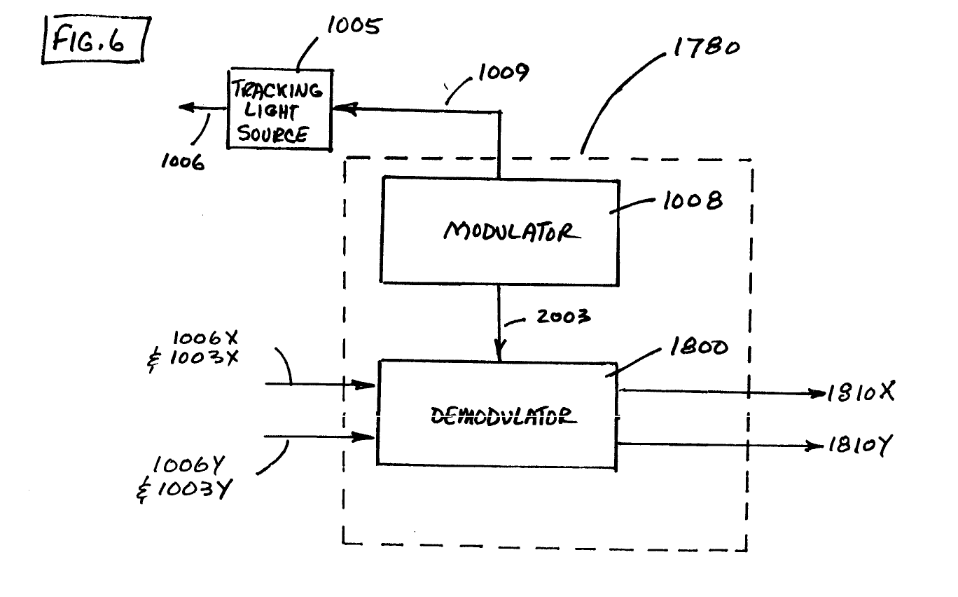

As is shown in FIG. 5a, the detector array 1600 comprises a plurality of detector elements 1620A-1620D. The detector elements 1620A and 1620C are positioned opposite each other on the X-axis 164. The detector elements 1620B and 1620D are positioned opposite each other on the Y-axis 163. In one embodiment, the detector elements 1620A-1620D each comprise a dual-element PIN silicon photodetector, such as the PIN SPOT-2DM1 manufactured by United Detector Technologies.

The light rays 1006F and 1003F each contribute to image 1670 of the tracking feature (e.g., the limbus 950) on the detector elements 1620A-1620D. The opposing pairs of detector elements 1620A/1620C and 1620B/1620D produce varying electrical outputs as the image 1670 of the tracking feature moves with respect to the X and Y axes. The arithmetic difference between signals from each pair of opposing detectors 1620A/1620C and 1620B/1620D is substantially proportional to the displacement of the image 1670 from the centered position in the corresponding axis.

When the cornea 930 of eye 900 is perfectly centered with respect to the axis 810, the image 1670 is centered on the detector elements 1620A-12620D, as shown in FIG. 5a. In this centered condition, the four detector elements 1620A-1620D receive essentially equal amounts of light energy from the image 1670. In this case, the detector array 1600 outputs voltage signals 1610 indicative of the centered condition.

When the cornea 930 is not perfectly (entered with respect to the axis 810, the image 1670 is not centered on the detectors 1620A-1620D; an example of which condition is shown in FIG. 5b. In this uncentered condition, at least two of the detector elements 1620A-1620D receive unequal amounts of light energy from the image 1670. In this case, the detector array 1600 outputs voltage signals 1610 that are proportional to the movement of the image 1670 relative to the axis 810.

It should be readily apparent from the foregoing to those skilled in the art that, in the absence of stray light 1003F, as the image 1670 of the tracking feature moves across the detectors 1620A-1620D, signals proportional to image displacement are produced as voltage signals 1006X and 1006Y. Once the image 1670 moves sufficiently for diametrically opposite detectors 1620A/1620C or 1620B/1620D to receive light only from the sclera 960 or the iris and not partially from both, the voltage signals 1610 cease to be linear with image displacement. The detector sizes can, however, be chosen so as to provide an appropriate linear range magnitude in both orthogonal directions, thereby ensuring dynamic tracking adequate to cover the anticipated lateral displacement of the cornea 930 in each direction.

As previously discussed, the highest accelerations of movements of the eye 900 occur during the saccades, so these eye motions would be the fastest and hardest to track. A typical saccade corresponds to a motion of up to about 5 degrees in 10 to 20 msec, which corresponds to about 1 mm of corneal translation, assuming a 1 in. diameter globe. Hence, the system 1 preferably is able to sense and respond to the eye's motion in 3-5 msec in order to provide real-time tracking. This corresponds to a response frequency of 200 to 300 Hz, which is 2-3 times faster than the eye and is easily achieved with standard electronics if the signal-to-noise ratio at the detector elements is high enough.

FIG. 6 illustrates the signal filtering action in more detail. As is seen there, the filter 1780 includes a modulator 1008 and a demodulator 1800 as well as signals 1009 to tracking light source 1005. The modulator 1008 outputs timing signals 2003 to the demodulator 1800, as well as signals 1009 to tracking light source 1005. The timing signals 2003 temporally synchronize the demodulator 1800 with the modulation frequency of the tracking light source 1005 used to illuminate the eye 900. This ensures that only light of an appropriate frequency is allowed to produce the tracking signals 1810X and 1810Y. As previously indicated, this synchronization constitutes a means for temporal discrimination of light 1006S used for tracking from light 1003S originating at ambient light sources 1002A and 1002B.

None of the prior art concerned with eye tracking has appreciated the unique advantages derived by modulating the light source 1005 so that the signals detected therefrom can be filtered from those due to other unwanted or stray light sources. Hence, a distinct advantage of the present invention over the prior art is clearly seen.

The diameter of the human eye limbus 950 is not constant for all the population; it typically varies, in adults, from approximately 10 to 14 mm. In some individual eyes, the vertical and horizontal dimensions of the limbus may differ slightly so the frontal aspect thereof may appear somewhat elliptical. Ideally, the rim of the image 1670 of the limbus 950 should be substantially coincident with the centers of the detectors 1620A-1620D in the array 1600 as indicated in FIG. 5a. For this to occur with varying limbus diameters, it is desirable to incorporate into the system a means for adjusting the size of the image 1670. This can be done by varying the optical magnification of the lens system forming the image 1670. While, theoretically, anamorphic magnification of the image could be provided so a slightly elliptical limbus could be aligned perfectly with the detector centers, this added complexity is not essential. Proper function of the present eye tracking system requires only that the rim of the limbus image 1670 be symmetrically disposed with respect to the centers of opposing detectors (1620A and 1620C in the vertical direction and 1620B and 1620D in the horizontal direction). Slight mismatch of image size in orthogonal directions due to an elliptical nature of the limbus image will not reduce the ability of the system to sense displacement of that image in each direction, and hence eye motion, as described above.

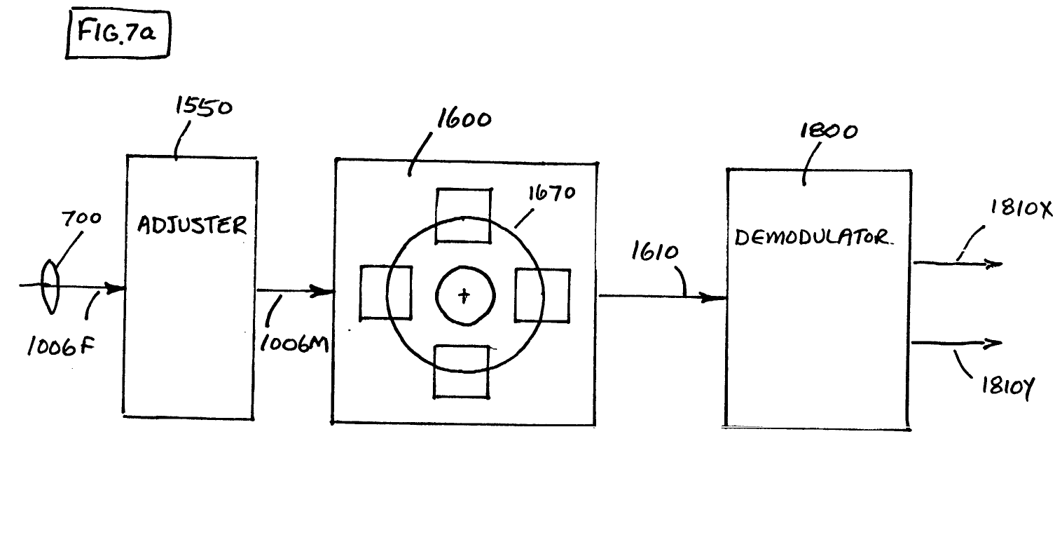

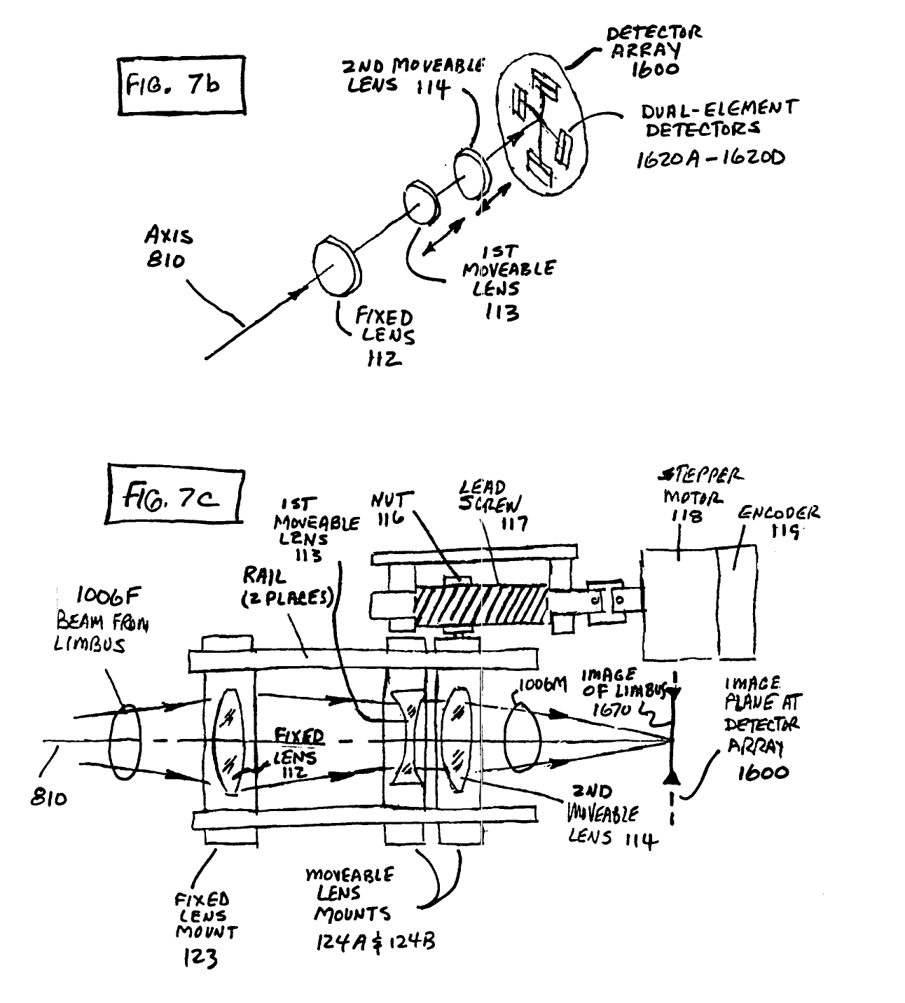

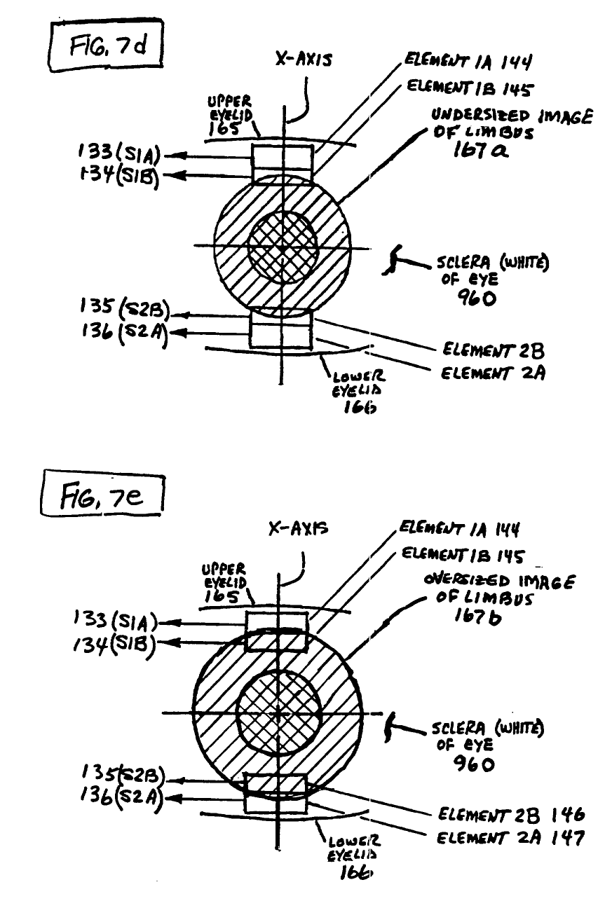

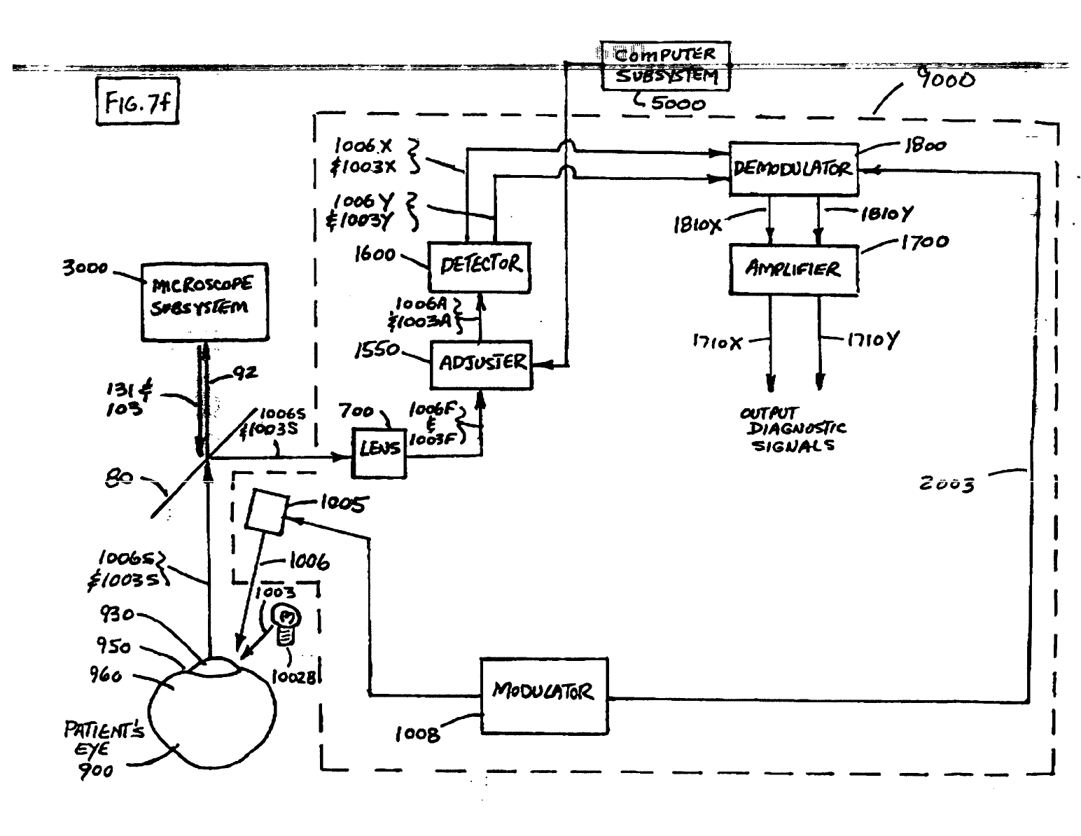

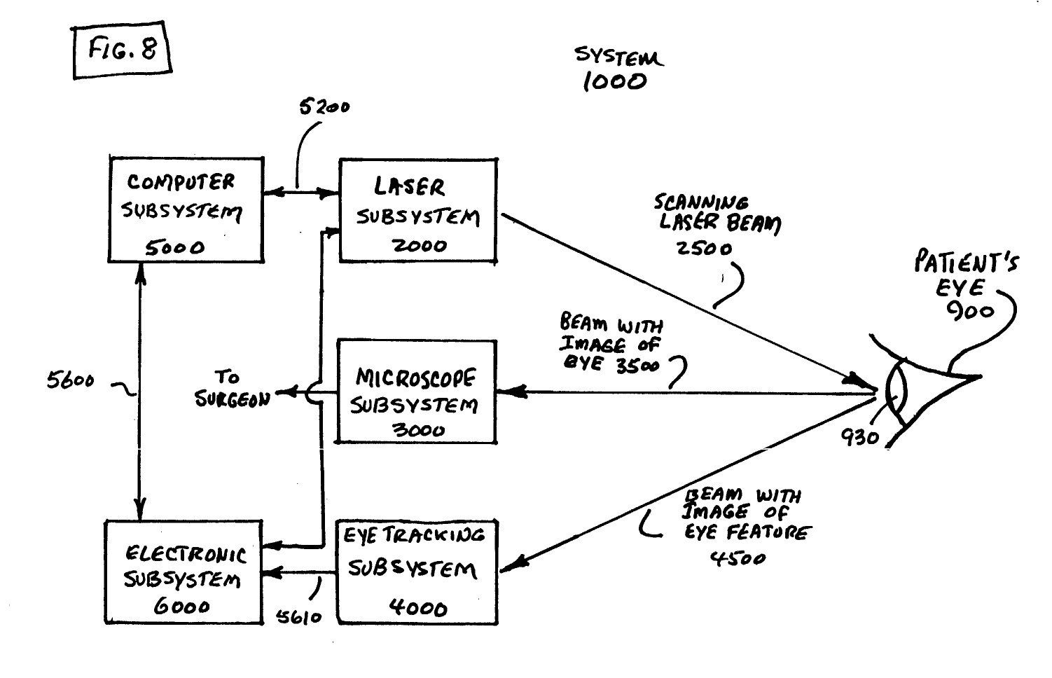

A means for varying the size of the image 1670 of the limbus 950 is described with reference to FIGS. 7a-7e. FIG. 7a depicts an adjuster 1550 that is positioned between the lens 700 and detector 1600 so as to receive the light rays 1006F. The adjuster 1550 introduces variable magnification into the beam comprising the light rays 1006F and outputs them in the form of rays 1006M as follows.

FIGS. 7b and 7c show one embodiment of an optical system that is used to vary magnification of the tracking feature image 1670 at the plane of the detector array 1600. Here, the image-forming component comprises a set of three refracting (lens) elements 112-114, at least two of which are axially moveable by external means such as a motorized drive mechanism. With two moving components, the magnification can be changed as required and sharp focus of the image 1670 at the detector array 1600 maintained. Note that the image-forming components of FIG. 7b may comprise single elements or multiple-elements, such as cemented doublets, for aberrational control reasons.

In one embodiment shown in FIG. 7c, each of the two moveable lens elements 113 and 114 is independently driven along axially-oriented tracks or rails by an electric motor 118 of the type commonly known as a “stepper” motor that turns a lead screw 117. A nut 116 attached to the moveable lens's mount (124a or 124b) engages said lead screw and moves along said screw as the screw is turned in a forward or reverse direction by the motor. Starting at a reference or “zero” position established by an encoder 119 attached to the motor 118, the motor 118 receives a series of pulsed drive signals from associated electronics (not shown). The motor 118 turns a fixed angular amount in response to each pulse received. In order to move the lens by a predetermined axial distance corresponding to a specific magnification change, the electronics delivers a corresponding specific number of pulses. An algorithm within a computer in communication with the encoder 119 may be used to control the electronics (not shown) driving both stepper motors so the movements of the two lenses 113 and 114 always remain synchronized.

One embodiment for automating the function of the magnification-change feature of this invention is described below. Following alignment of the patient to the axis 810, a computer routine is initiated that commands the magnification feature optics to adjust to a minimum value such that the image 1670 of the tracking feature 950 located at a predetermined distance in front of lens 700 will lie inside the centers of the detectors in array 1600 as shown in FIG. 7d. In this figure, the detectors 1620A/1620C are shown for the X-axis only for purposes of clarity. The geometry relating to the detectors 1620B/1620D in the Y axis would be similar to that shown. Each detector of FIGS. 7d and 7e is of the dual-element type as mentioned earlier. The magnification is increased and focus maintained under computer algorithm control while the electrical signals 133 (S1A), 134 (S1B), 135 (S2A), and 136 (S2B) are monitored. When the photometrically darker region of the image inside the limbus 950 reaches the junction between adjacent elements in each detector 1620A or 1620C, the signals from the outermost elements (S1A and S2A) will begin to decrease because progressively smaller areas of the image 1670 of the brighter-appearing sclera 960 will fall into those detector elements. When this change in signals is recognized by the computer, the stepper motors are stopped and electrically locked in place. The same condition would occur simultaneously in the Y-axis direction if the image of the limbus 950 is symmetrical, which is generally the case, and the condition shown in FIG. 7e would prevail. To allow for minor differences between end-points measured by the detector pairs in the X and Y axes, averages of the received signals can be derived and used by the computer. With the magnification now properly adjusted, tracking of the eye's motions can proceed as described earlier.

If the present invention were to be configured for tracking the pupil of the eye instead of the limbus thereof, this automatic magnification-adjusting feature would be advantageous in compensating for pupil diameter changes due to changes in illumination level and/or the effects of medication administered by the physician to facilitate the surgical procedure. The nominal magnification of the image-forming optics comprising lens 700, lens 112, lens 113, and lens 114 would need to be appropriately adjusted as would the dynamic range of the adjuster 1550.

A semiautomatic method for setting the magnification of system 1 also could be implemented as follows. Since the average limbus diameter of the patient's eye is easily measured during preparation for surgery, this dimension could be entered into an algorithm in a computer and the stepper motors 118 commanded to reposition the moveable lenses in accordance with a prior calibration to the proper locations to produce the properly sized, in-focus image of the limbus 950 at the detector array 1600. Once this adjustment is made, the stepper motors 118 can be electrically locked and tracking can proceed as described earlier.

FIG. 7f shows an embodiment of the tracker subsystem 9000 comprising tracking light 1005, modulator 1008, lens 700, adjuster 1550, detector 1600, demodulator 1800, and amplifier 1700 as it might be configured for use in tracking an object (here shown as eye 900) for use in, for example, a diagnostic application. Computer subsystem 5000 and microscope subsystem 3000 are depicted in their roles as means for controlling the magnification adjuster 1550 and observing the eye, respectively. The amplified output signals 1710X and 1710Y provide information as to the lateral movements of the cornea 930 relative to the line of sight of microscope 100 within microscope subsystem 3000. The beamsplitter 80 provides simultaneous optical access to the eye by the tracker and the microscope. Through the filtering action of modulator 1008 combined with tracking light 1005 and demodulator 1800 through connection 2003, these signals are not affected by the presence, absence, or nature of ambient light sources such as is represented by ambient light 1002B. This insensitivity to ambient illumination provides a distinct advantage of the present invention over prior art.

FIG. 8 depicts a system 1000 for surgical treatment of the eye with a laser into which the present invention has been integrated. The system 1000 includes a microscope subsystem 3000 through which a surgeon can view an eye 900 via beam 3500. Under the control of the surgeon who observes the eye 900 and the surgical treatment thereof through microscope subsystem 3000, the laser subsystem 2000 delivers a beam 2500 preferably comprising a sequence of short pulses of mid-infrared light to the cornea 930 of the eye 900. These pulses are moved over the cornea 930 in a predetermined pattern in accordance with predetermined commands from the computer subsystem 5000 that is coupled to the laser system 2000 through connection 5200.

Motions of the eye 900 during the surgical procedure are sensed by the tracking subsystem 4000 via a light beam 4500 that includes an image of the tracking feature of the eye 900. Commands to deviate the laser beam 2500 to compensate for such motions are delivered from the tracking subsystem 4000 to electronics subsystem 6000 via connection 5610, and then to the computer subsystem 5000 via connection 5600. The commands to deviate the laser beam 2500 are then delivered from the computer 5000 to the laser subsystem 2000 via connection 5200. In this way, the laser beam 2500 is deviated as required to center the pattern of laser pulses to the displaced cornea. The result is that the pattern of laser pulses at the eye 900 remains centered on the cornea as if the eye had not moved.

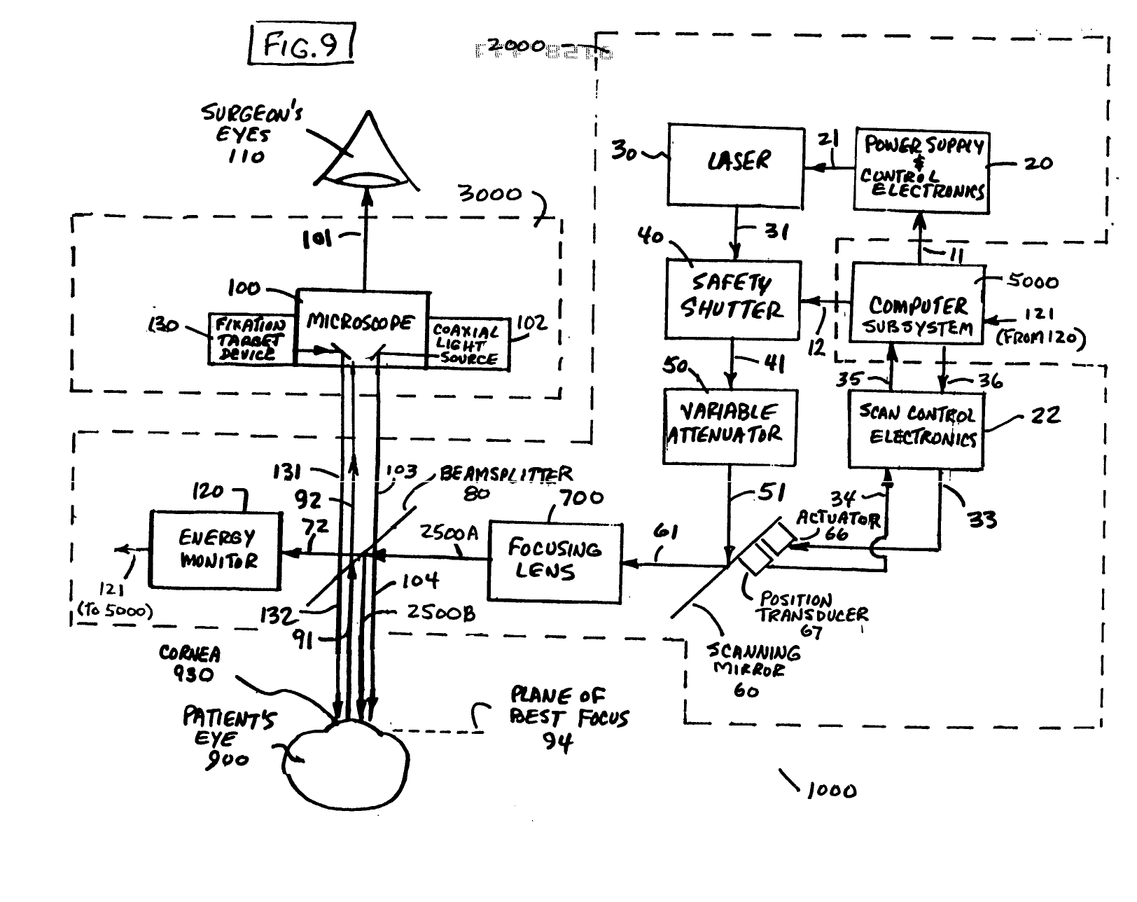

FIG. 9 is a block diagram of the system 1000 in which the laser subsystem 2000 and the microscope subsystem 3000 are shown in more detail and in relationship to the computer subsystem 5000. Commands 11 are sent to control 20 from computer 5000 to control the laser 30 via connection 21. The laser 30 is preferably a mid-infrared laser generating short laser pulses which yield a tissue removal mechanism based on photospallation as disclosed by Telfair et al in U.S. patent application Ser. No. 08/549,385.

The laser beam 31 passes through a safety shutter 40 as beam 41. The intensity of the beam 41 is controlled by variable attenuator 50 whose output is beam 51. The beam 51 is deviated through small angles in two orthogonal directions upon reflection from scanning mirror 60 to form beam 61. This beam 61 is focused by lens 700 into a small circular spot of laser light at the cornea 930 of the patient's eye 900 as 2500A. The lens 700 may comprise single or multiple refracting and/or reflecting elements.

The beam 2500A is incident upon and reflected by beamsplitter 80 as beam 2500B. The laser beam 2500B is preferably scanned over a specific centralized region of the surface of the cornea 930 in a predefined manner so as to selectively remove tissue at various points within the cornea 930 and thereby cause the curvature of the cornea 930 to change in a predictable and controlled fashion (PRK) or, in the case of a therapeutic intervention, to remove tissue substantially uniformly over the treated area (PTK).

The system 1000 also can be utilized to perform the procedure called LASIK in which controlled tissue removal occurs after a flap of anterior tissue has temporarily been lifted from the surface of the eye 900. By virtue of the scanning motion introduced by the mirror 60 as driven by a set of actuators 66 through connection set 33 to the scan control electronics 22, the focused beam 2500B traces a prescribed pattern on the cornea as directed by the computer 5000 via drive signals 36 and 33. Feedback as to the instantaneous position of the mirror 60 is given to the computer 5000 via a set of position transducers 67 and associated connection set 34 and 35. It should be noted that two actuators 66 (operating in push-pull fashion) and one transducer 67 are required for each axis of motion of the mirror 60.

Alignment of the eye 900 to the system 1000 is initially established and subsequently monitored by the surgeon who observes the eye 900 via reflected beams 91, 92, and 101 passing through beamsplitter 80 and magnified by microscope 100. The pulse energy monitor 120 measures the intensity of the laser beam 2500A via transmitted beam 72 and feeds this measurement to the control electronics 20 via connections 121 and 11 by way of computer 5000 to ensure that sufficient energy is deliverers to the eye 900 for the intended surgical procedure, but that safe limits on the energy are not exceeded.

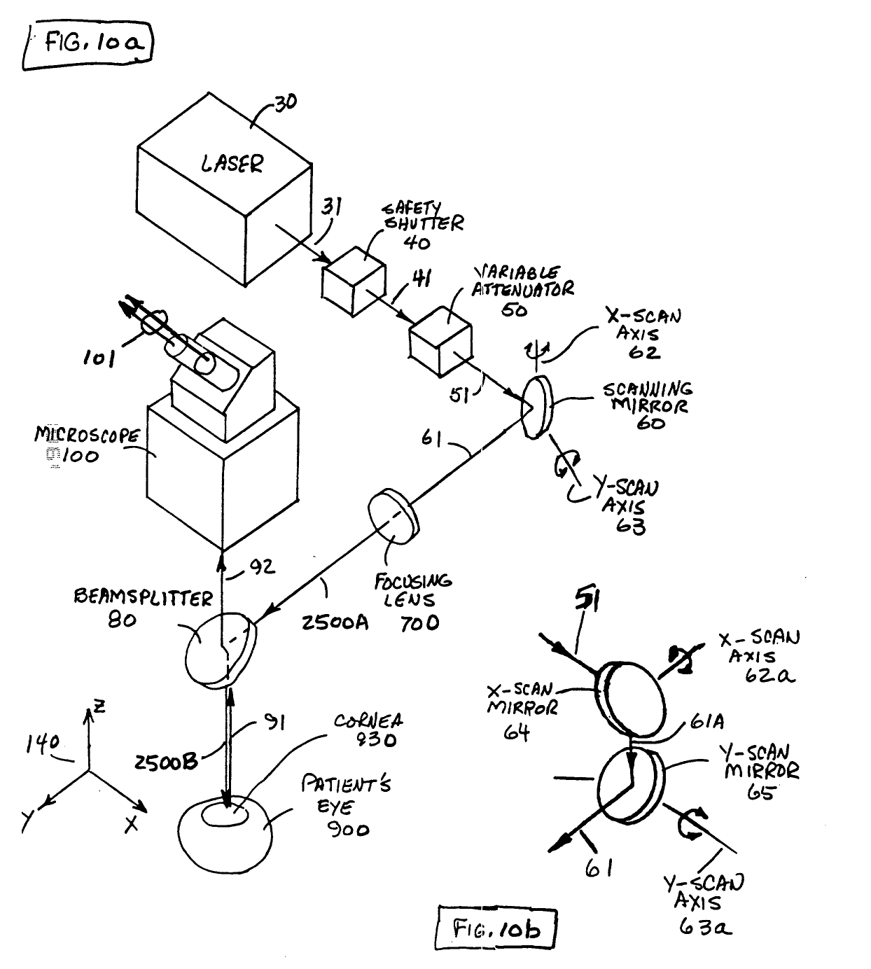

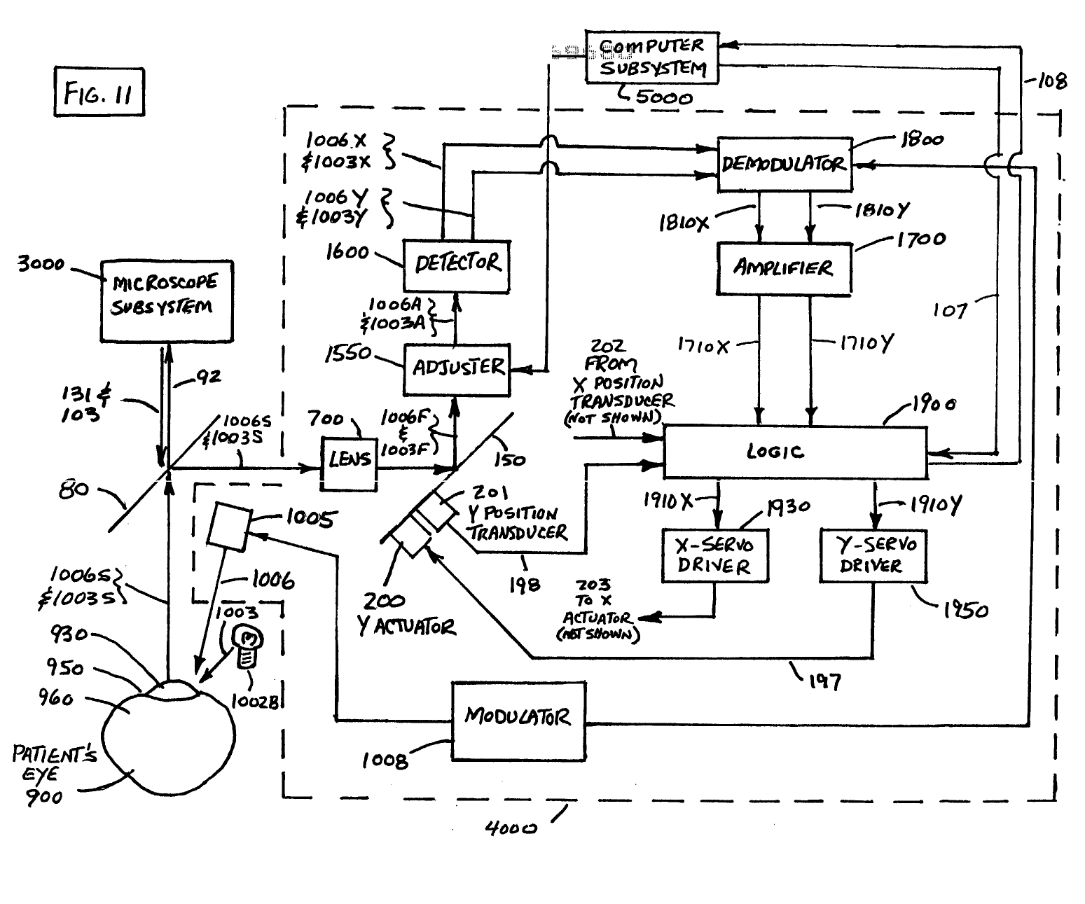

FIG. 10a illustrates schematically an assemblage of certain components from FIG. 9 in order to clarify their mutual spatial relationships. The scanning mirror 60 is shown as a two-axis gimbaled assembly which tilts about orthogonal axes 62 and 63 to affect movement of the focused spot of laser light at the cornea 930 in the coordinate system depicted as 140.

To correlate the reference frame of the eye 900 to that of the system 1000 as shown in FIGS. 9, 10a, and 10b, the line-of-sight of the patient's eye 900 is substantially coincident with the propagation axis of the undeviated incident laser beam 2500B. As used herein, in accordance with customary definition, the term “line-of-sight” or “principal line of vision” refers to the chief ray of the bundle of rays passing through the pupil of the eye 900 and reaching the fovea, thus connecting the fovea with the fixation point through the center of the entrance pupil. It will therefore be appreciated that the line-of-sight constitutes an eye metric defined directly by the patient, rather than through some external measurement of the eye's position and further, that the line of-sight can be defined without ambiguity for a given eye 900 and is the only axis amenable to objective measurement using cooperative patient fixation.

It is generally acknowledged that, for best post-surgery visual performance, the point marking the intersection of the line-of-sight with the cornea establishes the desired center for the optical zone of refractive procedures seeking to restore visual acuity. It is noted that the orientation of the line-of-sight of the eye 900, shown in FIGS. 9, 10a, and 10b, may be vertical, horizontal, or intermediate to those extremes as befitting comfortable positioning of the patient for surgery without affecting the effectiveness of the invention.

Visual access to the eye 900 by the surgeon's eyes through microscope 100 is by means of beamsplitter 80. The beamsplitter bears, on its side nearest to the eye 900, a thin-film coating that maximally reflects mid-infrared radiation in the wavelength region of 2.7 to 3.1 μm while partially transmitting visible light. The wavelength-preferential, or dichroic, nature of this coating serves to separate the functions, of the surgical laser 30 from that of the microscope 100 and, hence, to facilitate the surgeon's observation and control of the surgical process. The side of the beamsplitter 80 nearest to said microscope is conventionally anti-reflection coated to maximize transmission of visible light.

During preparation for laser surgery on the cornea 930, the line-of-sight of the eye 900 is aligned to coincide with the axis of the undeviated laser beam 2500B by two-axis lateral-translational adjustments, in a known manner, as directed by the surgeon. The surgeon observes the eye 900 by way of beams 91, 92, and 101 through the surgical microscope 100. In this way, the surgeon judges the degree of centration of the frontal image of the cornea 930 with respect to a crosshair or other fixed reference mark (not shown) internal to microscope 100 indicating, as a result of prior calibration, the location of the axis of undeviated laser beam 2500B.

The axial location of the cornea 930 can also be judged by the surgeon's eyes by virtue of the observed degree of focus of the image of corneal features relative to the previously calibrated and fixed object plane of best focus 94 for microscope 100. See FIG. 9. Directions from the surgeon allow adjustment of the axial position of the cornea of eye 90 to coincide with said plane of best focus 94.

We now describe in more detail the constituent parts of microscope subsystem 3000 as depicted in FIG. 9. Frontal illumination of the eye 900 to facilitate visual observation thereof by the surgeon viewing beams 101 exiting from microscope 100 in preparation for and during surgical procedures is provided by a light source 102 attached to or integral with the microscope 100. The light beam 103 from light source 102 typically emanates as beam 103 from a tungsten filament lamp therein and is incident upon the eye 900 as beam 104. The beams 103 and 104 propagate at a small angle, typically of the order of 0-10 degrees, with respect to the axis of the microscope 100. Such illumination is frequently termed coaxial or near-coaxial because of its angular proximity to that axis.

The angular orientation of the line-of-sight of eye 900 is preferably established by directing the patient to observe and focus attention, i.e., fixate, on beam 132 which is a continuation of beam 131 from an illuminated target (not shown) projected into the eye 900 by an optical fixation target device 130, which is preferably integrated into microscope 100 as indicated in FIG. 9. The target will appear to be located at a sufficient axial distance from the eye 900 of the patient so it can be observed and will have been previously aligned coaxially with the axis of the microscope 100.

As shown in FIGS. 9, 10a, and 10b, the laser subsystem 2000 preferably includes a safety shutter 40 which closes automatically if the laser beam 2500B fails to follow a prescribed path, if pulse energy-monitoring means 120 indicates a malfunction of laser 30, or if the eye tracker subsystem 4000 described below cannot adequately follow the eye motion (as might happen it the eye inadvertently moves beyond the dynamic range of the tracker). The surgeon also can close the shutter 40 by actuating a nearby emergency stop switch (not shown).

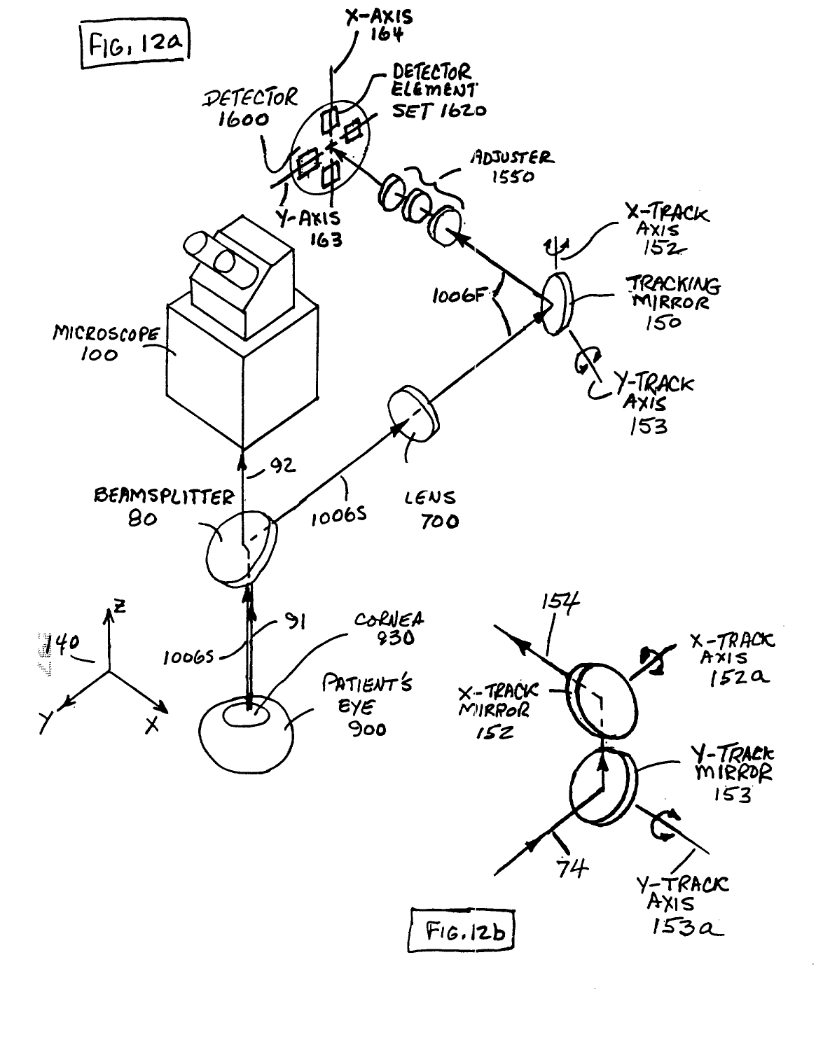

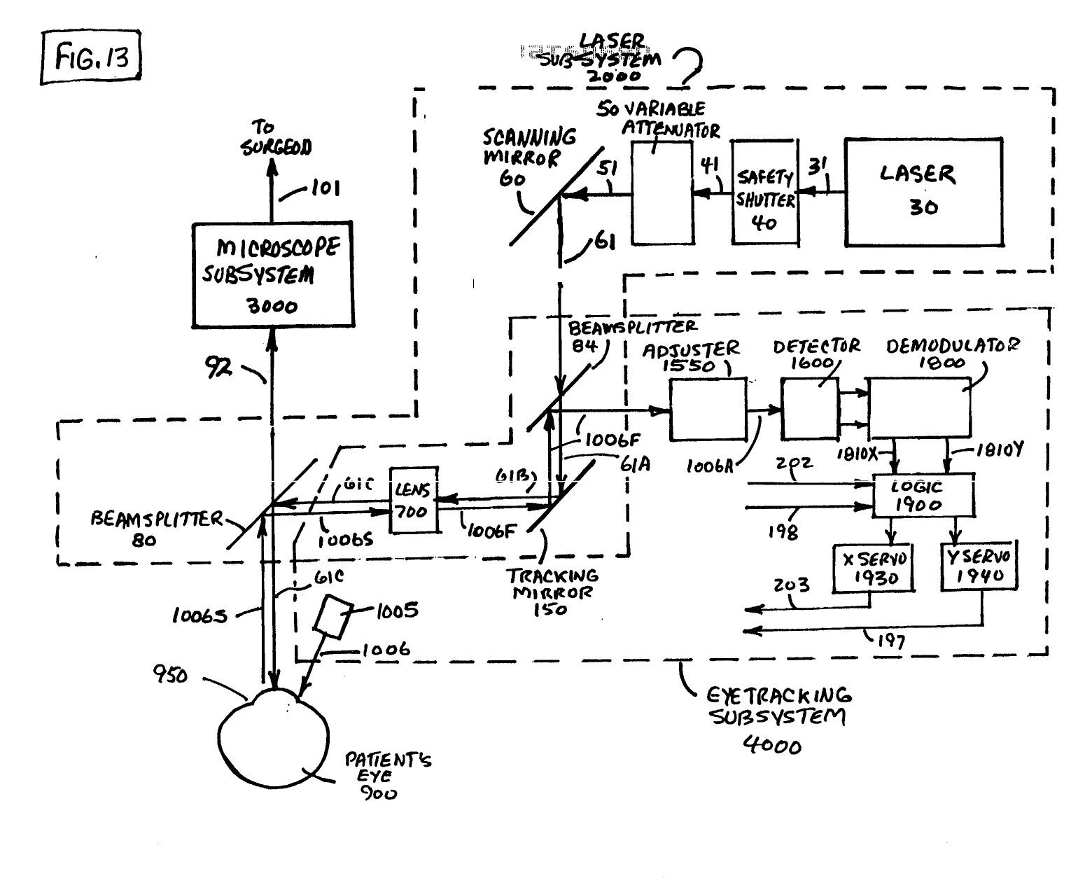

Lateral motions of the patient's cornea 930 (preferably less than about 5 mm in either orthogonal lateral direction X or Y) that occur after the initial alignment performed in the manner described above, or throughout the surgical treatment, are rendered inconsequential by virtue of the function of the eye tracker subsystem 4000 shown in more detail in FIGS. 11, 12a, and 12b. The eye tracker subsystem 4000 functions as described below to sense the motion of the cornea 930 and to provide electrical signals 1810X and 1810Y that are proportional to the lateral misalignment of the cornea 930 relative to the axis of the incident undeviated laser beam 2500B. The influence of stray light from ambient sources such as 1002B of FIG. 11 are here ignored because of the filtering action described earlier.

The signals 1810X and 1810Y are processed by demodulator 1800, amplifier set 1700, logic circuit 1900, and X- and Y-servo drivers 1930 and 1950 to cause tracking mirror 150 to restore centration of the image of cornea 930 formed by the lens 700 (and the magnification-adjusting lenses 1550 if used) at detector array 1600.

The eye tracking subsystem 4000 is integrated with the above-described laser system 2000 so any sensed eye movements can be quantitatively fed back to the laser system in such a manner as to compensate for the eye movements. This function is accomplished as indicated in FIG. 13. Scanned laser beam 61 is incident upon tracking mirror 150 as beam 61A after passing through beamsplitter 84. After reflection from the tracking mirror 150, the laser beam 61B passes through and is focused by lens 700 and proceeds to eye 900 as described above. An image of a selected feature of said eye, such as the limbus 950, is formed by the combined action of lens 700 and adjuster lenses 1550 located within eye tracking subsystem 4000 in front of detector 1600. This image is formed by a beam following the path 1006S, 1006F, 1006A by way of tracking mirror 15C and beamspliter 84. When the eye tracker subsystem 4000 senses and measures an eye movement, it sends signals 197 and 203 that cause tracking mirror 150 to tilt about its X and Y axes thereby compensating for the movement and reducing the signals 1810X and 1810Y to negligible values. Since the laser beam also reflects from tracking mirror 150, the reflected laser beam 61B and 61C is deflected so as to align the pattern of pulses caused by action of scanning mirror 60 to the center of the cornea 930. The surgical procedure therefore is accomplished as if the eye remained stationary.

It is noted, from FIG. 13, that beamsplitter 84, tracking mirror 150, lens 700, and beamsplitter 80 are common to both laser subsystem 2000 and eye tracker subsystem 4000. These components serve distinctive functions in each subsystem. For example, lens 700 focuses the mid-infrared laser beam 61B as beam 61C at or near the cornea and images the near-infrared beam 1006S bearing eye feature motion information into the adjuster 1550 and thence to detector 1600.

The functions of the single mirrors 60 (used for laser beam scanning) and 150 (used for eye tracking) as illustrated in FIGS. 9, 10a, 11, and 12a and 13, also could be accomplished by two mirrors independently scanning, about mutually orthogonal X and Y axes and intercepted in sequence by the laser beam enroute to the eye 900 as illustrated in FIGS. 10b and 12b. If two mirrors are used for scanning, the mirrors tilt as commanded by input X and Y drive signals 33 (shown in FIG. 9) about axes 62a and 63a (shown in FIG. 10b) to controllably move the reflected laser beam 61. Similarly, if two mirrors are used for tracking, the mirrors tilt as commanded by input X and Y drive signals 197 and 203 (shown in FIG. 11) about axes 152a and 153a (shown in FIG. 12b) to controllably move the reflected tracking beam 1006F.

Regardless as to whether single or double mirrors are used for scanning and tracking, the tracking mirror means (150 or 152 in combination with 153) are located closer to the patient's eye 900 than the scanning mirror (60 or 64 in combination with 65) so as to separate the functions thereof and to allow the scanned laser beam 2500B to be synchronized with measured movements of the eye 900.

It may be observed from FIGS. 9 and 10a, as well as FIGS. 11, and 12a in conjunction with FIG. 13, that the reflecting natures of tilting mirrors 60 (or 64 and 65 of FIG. 10b) and 150 (or 152 and 153 of FIG. 12b) play important roles when the present invention is used as part of the system 1000. In both cases, laser radiation in beams 51 and 61A is reflected. Near-infrared light in beam 1006F also is reflected by mirror 150 (or 152 and 153 of FIG. 12b). This can be accomplished through use of common aluminum or silver thin-film coatings protected by overcoats of suitable dielectric materials such as silicon monoxide. Multiple-layer dielectric coatings also could be employed for these purposes.

Similarly, the substrate of and coating on beamsplitter 84 of FIG. 13 would preferably be selected to have high transmittance at the mid-infrared wavelength of laser 30 and high reflectance at the visible and/or near-infrared wavelengths used by the eye tracker subsystem 4000. This dichroic coating, of a type frequently called a “cold mirror,” is commercially available from several suppliers, such as Optical Coating Laboratory, Inc., or Denton Vacuum, Inc. The other side of beamspliter 84 would preferably be antireflection coated for the wavelength of laser 30. The latter coating can be omitted if said beamsplitter is oriented at Brewster's angle of incidence for the wavelength of said laser 30.

Other arrangements of lenses, beamsplitters and mirrors could be incorporated into the optical system of this invention to accomplish the functions described herein. For example, beamsplitting prisms, typically in the form of cemented two-element cubes, each with a partially-reflecting, dichroic coating on an internal surface, might be employed to provide the functions of beamsplitters 80 and 84.

As shown in FIG. 13, at the beamsplilters 80 and 84 the transmitted beams 92 and 61A undergo small lateral displacements due to oblique incidence and the finite thickness of the component substrates. These fixed displacements are easily compensated for in the design of the apparatus, as would be apparent to a person of ordinary skill in the art.

As shown in FIG. 9, the computer subsystem 5000 communicates with and controls the laser source 30 through control 20 by means of connections 11 and 21. In addition, the computer 5000 provides commands to scan control electronics 22 via connection 36 which drives the scanning mirror 60 by means of connection 33 and a set of actuators 66 in accordance with stored scanning patterns and commands input to the computer 5000 by the surgeon or an assistant. A connection 12 between the computer 5000 and the safety shutter 40 provides means for affecting maximum safety of the patient, the surgeon, and attending personnel in the following manner. As shown in FIG, 11, the computer 5000 continually monitors the operation and status of the eye tracker subsystem 4000 by means of a connection 107 to the logic circuit 1900. If malfunction of the tracking mirror 150 occurs or if the signals 1710X and 1710Y received from detector 1600 through demodulator 1800 and amplifier 1700 fall outside allowable limits, the computer issues a command to close safety shutter 40 through the connection 12 (See FIG. 9). If monitor 120 senses laser energy outside predetermined limits, a signal 121 also commands computer subsystem 5000 to close shutter 40.

FIG. 11 also shows one embodiment of a servo system comprising detector 1600, demodulator 1800, amplifier set 1700, logic circuit 1900, X- and Y-servo drivers 1930 and 1950, actuator set 200 (X-axis not shown), and position transducer set 201 (X-axis not shown), as well as associated connections, used to drive the tracking mirror 150.

In one embodiment, the four detectors, collectively labeled set 1620 in FIG. 12a, each comprise a dual-element PIN silicon photodetector such as the PIN SPOT-2DM1 manufactured by United Detector Technologies. As indicated in FIG. 11, voltage signals 1006X and 1006Y, respectively, received from the detectors associated with the X- or Y-motion-sensing axis are sent to demodulator 1800 with the filtered signals 1810X and 1810Y then channeled directly into amplifier 1700.

The logic circuit 1900 converts the demodulated and amplified signals from the detector 1600 corresponding to limbus image position, into commands for controlling the tracking mirror 150. Diametrically opposing pairs of detectors 1620 produce varying electrical outputs as the image 1670 of the limbus 950 moves with respect to the X and Y axes.

The arithmetic difference between signals from each pair of opposing detectors is substantially proportional to the displacement of the image from the centered or null position in the corresponding axis. The signal differences produced within logic circuit 1900 and further processed by the logic circuit 1900 constitute mirror tilt commands indicated by control signals 1910X and 1910Y. The commands are relayed to the servo drivers 1930 and 1950 which, in turn, drive sets of actuators 200 which are mechanically linked to mirror 150, thus causing said mirror to pivot about one or both of its axes. In this manner, the angular orientation of the mirror 150 may be modified as required to follow the limbus image motion in two orthogonal lateral directions.

A set of transducers 201 are also mechanically connected to mirror 150 to provide feedback to logic circuit 1900 via connections 198 and 202 in the Y and X directions respectively. The transducers 201 generally comprise, position-sensing elements which, in one embodiment, are simple, readily-available capacitive sensors such as are made by Kaman Instrumentation Corp. In another embodiment, they may be optical encoders integral with actuator set 200. The transducers 201 facilitate stabilization of the motion of the tracking mirror 150, referenced to a pre-selected default position. In addition, the transducers 201 sense when the tracking mirror 150 is at the end of its, range and will no longer track the eye's motion. By connection 108, the logic 1900 commands the computer subsystem 5000 to close shutter 40, if the tracker is no longer able to follow the eye motion.

In one embodiment, the reference position of the mirror 150 corresponds to alignment of the patient's line-of-sight with the optical axes of the instrument and of the undeviated laser beam 2500B, as previously discussed. This reference position can be selected by the computer 5000, when the surgeon indicates that the patient's eye 900 is properly aligned.

The servo system shown in FIG. 11 preferably is an off-null measurement system based on returning the error signals to zero. There may be alternative implementations of a servo control system other than the one depicted in this figure which would allow the accurate measurement and/or control of eye displacements at sufficiently high rates.

Although the particular embodiments shown and described above will prove to be useful in many applications relating to the arts to which the present invention pertains, further modifications of the present invention herein disclosed will occur to persons skilled in the art. All such modifications are deemed to be within the scope and spirit of the present invention as defined by the appended claims.

We claim:

1. A system for facilitating tracking of a moving object, wherein the object has a feature associated therewith, wherein the feature is illuminated with ambient light, and wherein the system comprises:
(a) illumination means for illuminating at least the feature of the object with a tracking light;
(b) detection means for detecting an image of the feature and for outputting signals corresponding to movement of the image, wherein the signals have a first component due to the tracking light and a second component due to the ambient light;
(c) filter means for filtering the second component from the signals and for outputting the first component of the signals so that the ambient light is discriminated from the tracking light and the moving object can be tracked using the first component of the signals.

2. The system of claim 1, wherein the filter means comprises means for modulating the illumination means at a predefined frequency and means for demodulating the signals at the predefined frequency.

3. The system of claim 1, wherein the filter means comprises means for mechanically chopping the tracking illumination at a predefined frequency and means for demodulating the signals at the predefined frequency.

4. The system of claim 1, wherein the illumination means is for illuminating the feature from a substantially axial direction.

5. The system of claim 1, wherein the illumination means comprises a plurality of light sources for illuminating the feature from an off-axial direction such that light from one of the plurality of sources overlaps with light from adjacent sources.

6. The system of claim 1, wherein the illumination means comprises a light emitting diode.

7. The system of claim 1, wherein the illumination means comprises a diode laser.

8. The system of claim 1, further comprising means for adjusting the size of the image before the image is detected, and wherein the detection means is for detecting the adjusted image.

9. The system of claim 1, wherein the object is an eye and the feature of the object is the limbus.

10. The system of claim 1, wherein the object is an eye and the feature of the object is the pupil.

11. A method for facilitating tracking of a moving object, wherein the object has a feature associated therewith, wherein the feature is illuminated with ambient illumination, and wherein the method comprises:
(a) illuminating at least the feature of the object with a tracking illumination with an illumination means;
(b) generating an image of the feature using the tracking illumination;
(c) detecting the image and generating signals corresponding to movement of the image, wherein the signals have a first component due to the tracking illumination and a second component due to the ambient illumination;
(d) filtering the second component of the signals from the first component and outputting the first component so that the ambient illumination is discriminated from the tracking illumination and the moving object can be tracked using the first component of the signals.

12. The method of claim 11, wherein the step of filtering comprises the steps of modulating the illumination means at a predefined frequency and demodulating the signals at the predefined frequency.

13. The method of claim 11, wherein the step of filtering comprises mechanically chopping the tracking illumination at a predefined frequency and demodulating the signals at the predefined frequency.

14. The method of claim 11, wherein the step of illuminating comprises illuminating the feature from a substantially axial direction.

15. The method of claim 11, wherein the step of illuminating comprises illuminating the feature from an off-axial direction using a plurality of light sources such that light from one of the plurality of sources overlaps with light from adjacent sources.

16. The method of claim 11, wherein the step of illuminating comprises illuminating the feature using a light emitting diode.

17. The method of claim 11, wherein the step of illuminating comprises illuminating the feature using a diode laser.

18. The method of claim 11, further comprising the step of adjusting the size of the image before the image is detected, and wherein the step of detecting the image comprises the step of detecting the adjusted image.

19. The method of claim 11, wherein the object is an eye and the feature of the object is the limbus.

20. The method of claim 11, wherein the object is an eye and the feature of the object is the pupil.

21. A system for compensating for movement of an eye of a patient during a surgical procedure, wherein the eye has a feature and a visual axis associated therewith, wherein the feature is illuminated with ambient light, wherein the surgical procedure includes directing a laser beam upon the eye using a mirror, wherein the laser beam has an optical axis associated therewith, and wherein the system comprises:
(a) illumination means for illuminating at least the feature of the object with a tracking light;
(b) detection means for detecting an image of the feature and for outputting signals corresponding to movement of the image, wherein the signals have a first component due to the tracking light and a second component due to the ambient light;
(c) filter means for filtering the second component from the signals and for outputting the first component of the signals so that the ambient light is discriminated from the tracking light;
(d) logic means for receiving the filtered signals and for generating tracking signals based thereon; and
(e) means for directing the laser beam upon the eye based on the tracking signals to maintain a substantially centered condition between the optical axis of the laser beam and the visual axis of the eye.

22. The system of claim 21, wherein the filter means comprises means for modulating the illumination means at a predefined frequency and means for demodulating the signals at the predefined frequency.

23. The system of claim 21, wherein the filter means comprises means for mechanically chopping the tracking illumination at a predefined frequency and means for demodulating the signals at the predefined frequency.

24. The system of claim 21, wherein the illumination means is for illuminating the feature from a substantially axial direction.

25. The system of claim 21, wherein the illumination means comprises a plurality of light sources for illuminating the feature from an off-axial direction such that light from one of the plurality of sources overlaps with light from adjacent sources.

26. The system of claim 21, wherein the illumination means comprises a light emitting diode.

27. The system of claim 21, wherein the illumination means comprises a diode laser.

28. The system of claim 21, further comprising means for adjusting the image before the image is detected and wherein the detecting means is for detecting the adjusted image.

29. The system of claim 21, wherein the feature of the object is the limbus.

30. The system of claim 21, wherein the object is an eye and the feature of the object is the pupil.

31. A method for compensating for movement of an eye of a patient during a surgical procedure, wherein the eye has a feature and a visual axis associated therewith, wherein the feature is illuminated with ambient light, wherein the surgical procedure includes directing a laser beam upon the eye using a mirror, wherein the laser beam has an optical axis associated therewith, and wherein the system comprises:
(a) illuminating at least the feature of the object with a tracking illumination with an illumination means;
(b) generating an image of the feature using the tracking illumination;
(c) detecting the image and generating signals corresponding to movement of the image, wherein the signals have a first component due to the tracking illumination and a second component due to the ambient light;
(d) filtering the second component of the signals from the first component and outputting the first component so that the ambient light is discriminated from the tracking illumination;
(e) receiving the filtered signals and generating tracking signals based thereon; and
(f) directing the laser beam upon the eye based on the tracking signals to maintain a substantially centered condition between the optical axis of the laser beam and the visual axis of the eye.

32. The method of claim 31, wherein the step of filtering comprises the steps of modulating the illumination means at a predefined frequency and demodulating the signals at the predefined frequency.

33. The method of claim 31, wherein the step of filtering comprises mechanically chopping the tracking illumination at a predefined frequency and demodulating the signals at the predefined frequency.

34. The method of claim 31, wherein the step of illuminating comprises illuminating the feature from a substantially axial direction.

35. The method of claim 31, wherein the step of illuminating comprises illuminating the feature using a plurality of light sources from a off-axial direction such that light from one of the plurality of sources overlaps width light from adjacent sources.

36. The method of claim 31, wherein the step of illuminating comprises illuminating the feature using a light emitting diode.

37. The method of claim 31, wherein the step of illuminating comprises illuminating the feature using a diode laser.

38. The method of claim 31, further comprising the step of adjusting the image before the image is detected, and wherein the step of detecting the image comprises the step of detecting the adjusted image.

39. The method of claim 31, wherein the feature of the object is the limbus.

40. The method of claim 31, wherein the feature of the object is the pupil.

41. A system for compensating for movement of an eye of a patient during a surgical procedure, wherein the eye has a feature and a visual axis associated therewith, wherein the feature is illuminated with ambient light, wherein the surgical procedure includes directing a temporally-sequenced pattern of laser beam spots scanned across the eye using a mirror, wherein the laser beam pattern has an optical axis associated therewith, and wherein the system comprises:
(a) illumination means for illuminating at least the feature of the object with a tracking light;
(b) detection means for detecting an image of the feature and for outputting signals corresponding to movement of the image, wherein the signals have a first component due to the tracking light and a second component due to the ambient light;
(c) filter means for filtering the second component from the signals and for outputting the first component of the signals so that the ambient light is discriminated from the tracking light;
(d) logic means for receiving the filtered signals and for generating tracking signals based thereon; and
(e) means for directing the pattern of laser beam spots upon the eye based on the tracking signals to maintain a substantially centered condition between the optical axis of the pattern of laser beam spots and the visual axis of the eye.

42. The system of claim 41, wherein the filter means comprises means for modulating the illumination means at a predefined frequency and means for demodulating the signals at the predefined frequency.

43. The system of claim 41, wherein the filter means comprises means for mechanically chopping the tracking illumination at a predefined frequency and means for demodulating the signals at the predefined frequency.

44. The system of claim 41, wherein the illumination means is for illuminating the feature from a substantially axial direction.

45. The system of claim 41, wherein the illumination means comprises a plurality of light sources for illuminating the feature from an off-axial direction such that light from one of the plurality of sources overlaps with light from adjacent sources.

46. The system of claim 41, wherein the illumination means comprises a light emitting diode.

47. The system of claim 41, wherein the illumination means comprises a diode laser.

48. The system of claim 41, further comprising means for adjusting the image before the image is detected and wherein the detecting means is for detecting the adjusted image.

49. The system of claim 41, wherein the feature of the object is the limbus.

50. The system of claim 41, wherein the object is an eye and the feature of the object is the pupil.

51. The system of claim 41, wherein the beam-directing mirror comprises two mirrors, each directing the pattern of laser beam spots in relation to one of two orthogonal axes.

52. A method for compensating for movement of an eye of a patient during a surgical procedure, wherein the eye has a feature and a visual axis associated therewith, wherein the feature is illuminated with ambient light, wherein the surgical procedure includes directing a temporally-sequenced pattern of laser beam spots scanned across the eye using a mirror, wherein the laser beam pattern has an optical axis associated therewith, and wherein the system comprises:
(a) illuminating at least the feature of the object with a tracking illumination with an illumination means;
(b) generating an image of the feature using the tracking illumination;
(c) detecting the image and generating signals corresponding to movement of the image, wherein the signals have a first component due to the tracking illumination and a second component due to the ambient light;
(d) filtering the second component of the signals from the first component and outputting the first component so that the ambient light is discriminated from the tracking illumination;
(e) receiving the filtered signals and generating tracking signals based thereon; and
(f) directing the pattern of laser beam spots upon the eye based on the tracking signals to maintain a substantially centered condition between the optical axis of the pattern of laser beam spots and the visual axis of the eye.

53. The method of claim 52, wherein the step of filtering comprises the steps of modulating the illumination means at a predefined frequency and demodulating the signals at the predefined frequency.

54. The method of claim 52, wherein the step of filtering comprises mechanically chopping the tracking illumination at a predefined frequency and demodulating the signals at the predefined frequency.

55. The method of claim 52, wherein the step of illuminating comprises illuminating the feature from a substantially axial direction.

56. The method of claim 52, wherein the step of illuminating comprises illuminating the feature using a plurality of light sources from a off-axial direction such that light from one of the plurality of sources overlaps with light from adjacent sources.

57. The method of claim 52, wherein the step of illuminating comprises illuminating the feature using one or more light emitting diode(s).

58. The method of claim 52, wherein the step of illuminating comprises illuminating the feature using one or more diode laser(s).

59. The method of claim 52, further comprising the step of adjusting the image before the image is detected, and wherein the step of detecting the image comprises the step of detecting the adjusted image.

60. The method of claim 52, wherein the feature of the object is the limbus.

61. The method of claim 52, wherein the feature of the object is the pupil.

62. The method of claim 52, wherein the beam-directing mirror comprises two mirrors, each directing the pattern of laser beam spots in relation to one of two orthogonal axes.
